(19) United States
(12) Patent Application Publication
(10) Pub. No.: US 2004/0058344 A1
(43) Pub. Date: Mar. 25, 2004

(54) Trans-splicing mediated imaging of gene expression

(76) Inventors: Lloyd G. Mitchell, Bethesda, MD (US); M. Puttaraju, Germantown, MD (US); S. Gary Mansfield, Montogomery Village, MD (US)

Correspondence Address: BAKER & BOTTS, 30 ROCKEFELLER PLAZA, NEW YORK, NY, 10112

(21) Appl. No.: 10/374,784

(22) Filed: Feb. 25, 2003

Related U.S. Application Data
(60) Provisional application No. 60/359,948, filed on Feb. 25, 2002.

(51) Int. Cl. C12Q 1/68; C12P 21/02; C12N 5/06; C12N 15/85

(52) U.S. Cl. 435/6; 435/69.1; 435/455; 435/320.1; 435/325

(57) ABSTRACT

The present invention provides methods and compositions for imaging of gene expression in cells. The compositions of the invention include pre-trans-splicing molecules (PTMs) designed to interact with a target precursor messenger RNA molecule (target pre-mRNA) expressed within a cell and mediate a trans-splicing reaction resulting in the generation of a novel chimeric RNA molecule (chimeric RNA) capable of encoding a reporter molecule. The PTMs of the invention are designed to interact with target pre-mRNAs thereby providing a method for detection of target pre-mRNA expression. The methods and compositions of the invention may be utilized to monitor the expression of specific genes within a cell. In instances where specific gene expression is associated with a disease, the present invention provides diagnostic methods and compositions. Such diseases include infectious diseases, proliferative disorders such as cancer, genetic, neurological and metabolic disorders, to name a few. Additionally, the present invention may be used in screening assays to identify compounds capable of modulating gene expression or in assays designed to identify protein/protein interactions. The invention is demonstrated by way of example in which papilloma virus gene expression within a cell was detected using a bioluminescence assay system.

### INTRODUCTION

The present invention provides methods and compositions for imaging of gene expression in cells. The compositions of the invention include pre-trans-splicing molecules (PTMs) designed to interact with a target precursor messenger RNA molecule (target pre-mRNA) expressed within a cell and mediate a trans-splicing reaction resulting in the generation of a novel chimeric RNA molecule (chimeric RNA) capable of encoding a reporter molecule. The PTMs of the invention are designed to interact with target pre-mRNAs thereby providing a method for detection of target pre-mRNA expression. The methods and compositions of the invention may be utilized to monitor the expression of specific genes within a cell. In instances where specific gene expression is associated with a disease, the present invention provides diagnostic methods and compositions. Such diseases include infectious diseases, proliferative disorders such as cancer, genetic, neurological and metabolic disorders, to name a few. Additionally, the present invention may be used in screening assays to identify compounds capable of modulating gene expression or in assays designed to identify protein/protein interactions. The invention is demonstrated by way of example in which papilloma virus gene expression within a cell was detected using a bioluminescence assay system.

### BACKGROUND OF THE INVENTION

DNA sequences in the chromosome are transcribed into pre-mRNAs which contain coding regions (exons) and generally also contain intervening non-coding regions (introns). Introns are removed from pre-mRNAs in a precise process referred to as splicing. In most cases, the splicing reaction occurs within the same pre-mRNA molecule, which is termed cis-splicing. Splicing between two independently transcribed pre-mRNAs is termed trans-splicing. Trans-splicing was first discovered in trypanosomes (Sutton & Boothroyd, 1986, Cell 47:527; Murphy et al., 1986, Cell 47:517) and subsequently in nematodes (Krause & Hirsh, 1987, Cell 49:753); flatworms (Rajkovic et al., 1990, Proc. Nat'l. Acad. Sci. USA, 87:8879; Davis et al., 1995, J. Biol. Chem. 270:21813) and in plant mitochondria (Malek et al., 1997, Proc. Nat'l. Acad. Sci. USA 94:553). In the parasite Trypanosoma brucei, all mRNAs acquire a splice leader (SL) RNA at their 5′ termini by trans-splicing. A 5′ leader sequence is also trans-spliced onto some genes in Caenorhabditis elegans. This mechanism is appropriate for adding a single common sequence to many different transcripts.

The mechanism of spliced leader trans-splicing, which is nearly identical to that of conventional cis-splicing, proceeds via two phosphoryl transfer reactions. The first causes the formation of a 2′-5′ phosphodiester bond producing a ‘Y’ shaped branched intermediate, equivalent to the lariat intermediate in cis-splicing. The second reaction, exon ligation, proceeds as in conventional cis-splicing. In addition, sequences at the 3′ splice site and some of the snRNPs which catalyze the trans-splicing reaction, closely resemble their counterparts involved in cis-splicing.

Trans-splicing may also refer to a different process, where an intron of one pre-mRNA interacts with an intron of a second pre-mRNA, enhancing the recombination of splice sites between two conventional pre-mRNAs. This type of trans-splicing was postulated to account for transcripts encoding a human immunoglobulin variable region sequence linked to the endogenous constant region in a transgenic mouse (Shimizu et al., 1989, Proc. Nat'l. Acad. Sci. USA 86:8020). In addition, trans-splicing of c-myb pre-RNA has been demonstrated (Vellard, M. et al. Proc. Nat'l. Acad. Sci., 1992 89:2511-2515), trans-spliced RNA transcripts from SV40 have been detected in cultured cells and nuclear extracts (Eul et al., 1995, EMBO. J. 14:3226) and more recently, the transcript from the p450 gene in human liver has been shown to be trans-spliced (Finta et al., 2002, J. Biol Chem 22:5882-5890). However, in general, naturally occurring trans-splicing of mammalian pre-mRNAs is thought to be an exceedingly rare event.

In vitro trans-splicing has been used as a model system to examine the mechanism of splicing by several groups (Konarska & Sharp, 1985, Cell 46:165-171 Solnick, 1985, Cell 42:157; Chiara & Reed, 1995, Nature 375:510; Pasman and Garcia-Blanco, 1996, Nucleic Acids Res. 24:1638). Reasonably efficient trans-splicing (30% of cis-spliced analog) was achieved between RNAs capable of base pairing to each other, splicing of RNAs not tethered by base pairing was further diminished by a factor of 10. Other in vitro trans-splicing reactions not requiring obvious RNA-RNA interactions among the substrates were observed by Chiara & Reed (1995, Nature 375:510), Bruzik J. P. & Maniatis, T. (1992, Nature 360:692) and Bruzik J. P. and Maniatis, T., (1995, Proc. Nat'l. Acad. Sci. USA 92:7056-7059). These reactions occur at relatively low frequencies and require specialized elements, such as a downstream 5′ splice site or exonic splicing enhancers.

U.S. Pat. Nos. 6,083,702, 6,013,487 and 6,280,978 describe the use of PTMs to mediate a trans-splicing reaction by contacting a target precursor mRNA to generate novel chimeric RNAs. The resulting RNA can encode any gene product including a protein of therapeutic value to the cell or host organism, a toxin, such as Diptheria toxin subunit A, which causes killing of the specific cells or a novel protein not normally present in cells. The PTMs can also be engineered for the identification of exon/intron boundaries of pre-mRNA molecules using an exon tagging method and for production of chimeric proteins including those encoding peptide affinity purification tags which can be used to purify and identify proteins expressed in a specific cell type.

Recent advances in molecular techniques have resulted in an increased understanding of the molecular basis of gene expression. In many disorders or diseases the expression of specific genes can be correlated with the presence of that disorder or disease. Thus, methods designed to detect and/or quantify such gene expression provide useful tools for studying gene expression within the cell, identifying compounds capable of modulating gene expression, and diagnosing disease in a subject. The present invention provides a powerful new tool for detecting the expression of a specific target gene within a living cell in real time.

### SUMMARY OF THE INVENTION

The present invention provides methods and compositions for imaging of gene expression within cells. The compositions of the invention comprise pre-trans-splicing molecules (PTMs) engineered to express a reporter molecule and the use of such molecules to detect the expression of specific genes within a cell. Such reporter molecules include but are not limited to fluorescent and bioluminescenct molecules, enzymes, receptors and peptide tags.

The methods and compositions of the invention provide a mechanism for targeting expression of a reporter gene product to a specific cell type. The methods of the invention encompass contacting the PTMs of the invention with a cell under conditions in which a portion of the PTM is trans-spliced to a portion of the target pre-mRNA to form a chimeric RNA. The target pre-mRNA may be any pre-mRNA expressed in any type of cell, including plant cells, where the goal is to detect expression of the pre-mRNA. Such cell types may include, but are not limited to those infected with viral or other infectious agents, benign or malignant neoplasms, cells expressing components of the immune system which are involved in autoimmune disease or tissue rejection or those cells expressing any target pre-mRNA known to be associated with a disease. In addition, the present invention provides screening assays designed to identify compounds capable of modulating gene expression or assays designed to identify protein/protein interactions.

### BRIEF DESCRIPTION OF THE DRAWINGS

FIG. 1. Schematic representation of different trans-splicing reactions. (a) Trans-splicing reactions between the target 5′ splice site and PTMs 3′ splice site; (b) trans-splicing reactions between the target 3′ splice site and PTM's 5′ splice site and (c) replacement of internal exon by double trans-splicing reaction in which the PTM carry both 3′ and 5′ splice sites. BD, binding domains; BP, branchpoint sequence; PPT, polypyrimidine tract and ss, splice sites.

FIG. 2. Schematic diagrams of the pre-mRNA targets; (a) HPV type 16 (b) β-HCG6 and (c) EGFR.

FIG. 3. Schematic diagrams of a prototype PTM and splice mutants showing the important structural elements of trans-splicing domain. BD, binding domain; BP, branchpoint and PPT, polypyrimidine tract. Unique restriction sites in the trans-splicing domain are indicated.

FIG. 4. Illustration of safety mechanism. (a) Schematic diagram of the safety PTM showing the intra-molecular base-paired stem-loop structure designed to cover the 3′ splice elements from splicing factors. (b) Diagram of a safety PTM in open configuration after binding to the β-HCG6 pre-mRNA target.

FIG. 5A. Trans-splicing mediated mRNA repair and production of functional protein. FIG. 5B. In situ staining for β-Gal activity following co-transfection in 293T cells (unselected). Cells transfected with (a) defective lacZ target alone, and (b) co-transfected with target and PTM.

FIG. 6. Pre-mRNA target that is designed to express part of the synthetic Renilla luciferase sequence, coupled to the coding sequences for E7 and sequences immediately upstream of E7 from the human papilloma virus (HPV).

FIG. 7. Pre-trans-splicing molecule (PTM) designed to base pair with the target intron and trans-splice in the 3′ luciferase “exon.”

FIG. 8. Repair model showing the binding of PTM to the target pre-mRNA and restoration of luciferase activity by trans-splicing.

FIG. 9. RT-PCR analysis of total RNA using target and PTM specific primers that produced the expected trans-spliced (435 bp) product only in cells that contain both target and PTM but not in controls (target, PTM alone and target+splice mutant PTM).

FIG. 10. Direct sequencing of the RT-PCR product confirms the accurate trans-splicing between the target and PTM.

FIG. 11. Co-transfection of a specific target with Luc-PTM13 resulted in the repair and restoration of Renilla luciferase function that is on the order of 4-logs over background. No luciferase activity above background was detected in controls or with splice mutant PTMs suggesting that the restoration of luciferase function is due to trans-splicing.

FIG. 12. Schematic drawings of Luc-PTM13, Luc-PTM14 and the splice mutant used for the study.

FIG. 13. Repair of human papilloma virus target pre-mRNA by trans-splicing in HEK293T cells.

FIG. 14. Repair of human papilloma virus target pre-mRNA by trans-splicing and restoration of luciferase function in HEK293T cells.

FIG. 15. Schematic of luciferase firefly pre-trans-splicing molecules.

FIG. 16. Trans-splicing strategy to monitor the expression of human papilloma virus.

FIG. 17. Luciferase expression with and without target.

FIG. 18. Schematic of Renilla luciferase pre-trans-splicing molecule.

FIG. 19. Trans-splicing strategy to monitor the expression of human papilloma virus employs Renilla 5′ “exon” replacement.

FIG. 20. Schematic diagrams of hemi-reporter model targets and PTMs used for imaging of gene expression. The mini-gene pre-mRNA targets consisting of 5′ portion of humanized Renilla luciferase (hRluc) to act as a “5′ exon” coupled to the E6-E7 intron region and adjacent E7 coding sequence of human papilloma virus (HPV16).

FIG. 21. Evaluation of trans-splicing efficiency at the RNA level.

FIG. 22. Evaluation of trans-splicing efficiency at the functional level. The efficiency of trans-splicing mediated mRNA repair and restoration of Luciferase function was confirmed by assaying for enzymatic activity.

FIGS. 23A-B. In vivo imaging using trans-splicing. The full length imaging PTM (Luc-PTM27) contains the complete coding sequence for humanized Renilla Luciferase (hRL) minus the AUG start codon. The trans-splicing domain consists of a strong 3′ splice element (including a yeast consensus branch point (BP), a long pyrimidine tract (PPT) and a 3′ acceptor site), a spacer sequence and a 80 nucleotide binding domain (BD) complementary to the 3′ end of the intron between exons E6 and E7 of human papilloma virus (HPV-16) (FIG. 23A). Schematic illustration of trans-splicing mediated restoration of Luciferase function is shown in FIG. 23B.

FIG. 24. Trans-splicing mediated mRNA repair and restoration of hRenilla Luciferase activity in 293T cells.

FIGS. 25A-B. Luciferase splice mutant PTM constructed to determine whether the restoration of Luciferase function is due to RNA trans-splicing. FIG. 25A, structure of a full-length imaging PTM (functional PTM); FIG. 25B, structure of a splice-mutant PTM. The splice mutant PTM is a derivative of Luc-PTM38 in which the 3′ splice elements such as BP, PPT and the acceptor AG dinucleotide were modified by PCR mutagenesis and were confirmed by sequencing.

FIG. 26. Restoration of Luciferase function is due to RNA trans-splicing.

FIG. 27. In vivo imaging of gene expression.

FIG. 28. In vivo imaging of gene expression following IV PTM delivery.

### DETAILED DESCRIPTION OF THE INVENTION

The present invention provides methods and compositions for imaging of gene expression in live cells. The compositions of the invention comprise pre-trans-splicing molecules (PTMs) engineered to express a reporter molecule and the use of such molecules for generating novel nucleic acid molecules. The PTMs of the invention are engineered to interact with target pre-mRNAs where the intention is to detect target pre-mRNA expression within a cell. The PTMs of the invention comprise one or more target binding domains that are designed to specifically bind to a target pre-mRNA, a 3′ splice region that includes a 3′ splice acceptor site and/or a 5′ splice donor site. The PTM may further comprise a branchpoint, a pyrimidine tract and one or more spacer regions that separate the splice sites from the target-binding domain.

In addition, the PTMs of the invention are engineered to contain any nucleotide sequence encoding a protein product that functions as a reporter molecule. Such reporter molecules include but are not limited to fluorescent and biluminescent molecules, enzymes, receptors and protein/peptide tags, antibodies or fragments thereof, and ion channel or subunits thereof. In some instances, the reporter molecule itself may provide the detectable signal, while in other cases a reporter probe, or tracer, having an affinity for the reporter molecule will provide the detectable signal, i.e., fluorescence, bioluminescence or radioactive label.

The methods of the invention encompass contacting the PTMs of the invention with a specific target pre-mRNA expressed within a cell under conditions in which a portion of the PTM is trans-spliced to a portion of the target pre-mRNA to form a chimeric RNA capable of encoding a reporter molecule. The target pre-mRNA may be selected because its expression is associated with a specific disease thus providing a mechanism for diagnosing the presence of disease in a subject. Such diseases may include, for example proliferative disorders such as benign or malignant neoplasms, genetic, metabolic, neurological or immunological disorders. For example, components of the immune system which are involved in autoimmune disease or tissue rejection may be targeted. Additionally, cells infected with viruses or other types of infectious agents may be targeted. The present invention also provides screening methods designed to identify agents capable of modulating gene expression. Alternatively, the methods and compositions of the invention may be utilized to identify the repertoire of mRNAs expressed within a specific cell type or to identify protein/protein interactions.

Structure of the Pre-Trans-Splicing Molecules

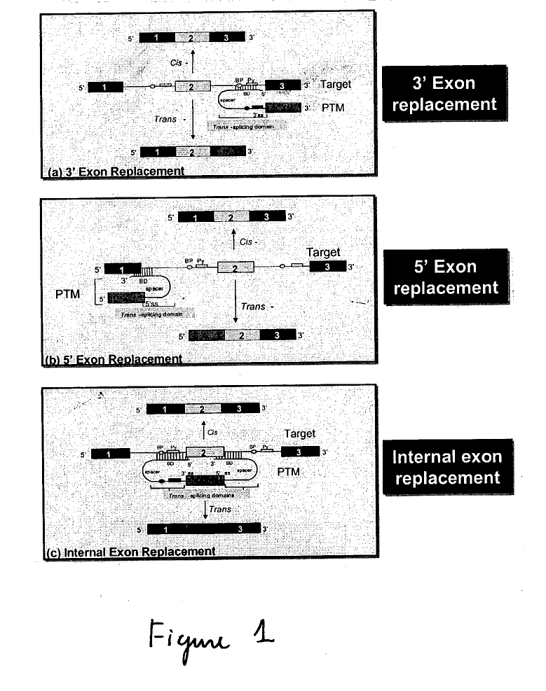

The present invention provides compositions for use in generating novel chimeric nucleic acid molecules encoding a reporter molecule through targeted trans-splicing. FIG. 1 is a schematic representation of the different types of trans-splicing reactions. The PTMs of the invention comprise (i) one or more target binding domains that targets binding of the PTM to a pre-mRNA target (ii) a 3′ splice region that includes a 3′ splice acceptor site and/or 5′ splice donor site; and (iii) a nucleotide capable of encoding a reporter molecule. In some instances, the PTMs of the invention may be engineered with no target binding domain or randomized target binding domains and/or a safety sequence. Additionally, the PTMs of the invention may further comprise one or more spacer regions that separate the RNA splice site from the target binding domains.

The target-binding domain of the PTM may contain multiple binding domains which are complementary to and in anti-sense orientation to the targeted region of the selected pre-mRNA. As used herein, a target binding domain(s) is defined as any sequence that confers specificity of binding and anchors the pre-mRNA closely in space so that the spliceosome processing machinery of the nucleus can trans-splice a portion of the PTM to a portion of the pre-mRNA. The target binding domains may comprise up to several thousand nucleotides. In preferred embodiments of the invention the binding domains may comprise at least 10 to 30 and up to several hundred nucleotides. The specificity of the PTM may be increased significantly by increasing the length of the target binding domain. In addition, although the target binding domain may be “linear” it is understood that the RNA may fold to form secondary structures that may stabilize the complex by preventing activation of the PTM splice site until the binding domain has encountered its target thereby increasing the specificity of trans-splicing. A second target binding region may be placed at the 3′ end of the molecule and can be incorporated into the PTM of the invention. Absolute complementarity, although preferred, is not required. A sequence “complementary” to a portion of an RNA, as referred to herein, means a sequence having sufficient complementarity to be able to hybridize with the RNA, forming a stable duplex. The ability to hybridize will depend on both the degree of complementarity and the length of the nucleic acid (See, for example, Sambrook et al., 1989, Molecular Cloning, A Laboratory Manual, 2d Ed., Cold Spring Harbor Laboratory Press, Cold Spring Harbor, N.Y.). Generally, the longer the hybridizing nucleic acid, the more base mismatches with an RNA it may contain and still form a stable duplex. One skilled in the art can ascertain a tolerable degree of mismatch or length of duplex by use of standard procedures to determine the stability of the hybridized complex.

In an embodiment of the invention, the target binding domain of the PTM will contain sequences which are complementary to and in anti-sense orientation to target pre-mRNA molecules where the goal is to detect expression of the target pre-mRNA. For example, PTM binding sites may be engineered to bind to any target pre-mRNA where the expression of the target pre-mRNA is associated with a disorder or disease. Such disorders include but are not limited to proliferative, immunological, metabolic or genetic disorders. In a specific embodiment of the invention, the β-chorionic gonadotropin 6 pre-target mRNA and/or the epidermal growth factor receptor pre-target mRNA, both known to be over expressed in tumor cells, may be targeted to detect proliferative disorders. Alternatively, the target binding domain of the PTM will contain sequences complementary to pre-mRNA molecules encoded for by an infectious agent. For example, target binding domains may be designed to bind to viral, bacterial or parasitic pre-mRNAs thereby providing diagnostic methods for detection of infectious diseases. FIG. 2 is a schematic diagram of HPV-16, β-HCG6 and EGFR pre-mRNA targets that may be used.

For screening assays designed to identify agents capable of modulating the expression of a particular gene of interest, the target binding domain of the PTM will contain sequences complementary to the gene of interest.

Binding may also be achieved through other mechanisms, for example, through triple helix formation or protein/nucleic acid interactions such as those in which the PTM is engineered to recognize a specific RNA binding protein, i.e., a protein bound to a specific target pre-mRNA. Alternatively, the PTMs of the invention may be designed to recognize secondary structures, such as for example, hairpin structures resulting from intramolecular base pairing between nucleotides within an RNA molecule.

The PTM molecule also contains a 3′ splice region that may include a branchpoint, pyrimidine tract and a 3′ splice acceptor AG site and/or a 5′ splice donor site. Consensus sequences for the 5′ splice donor site and the 3′ splice region used in RNA splicing are well known in the art (See, Moore, et al., 1993, The RNA World, Cold Spring Harbor Laboratory Press, p. 303-358). In addition, modified consensus sequences that maintain the ability to function as 5′ donor splice sites and 3′ splice regions may be used in the practice of the invention. Briefly, the 5′ splice site consensus sequence is AG/GURAGU (where A=adenosine, U=uracil, G=guanine, C=cytosine, R=purine and /=the splice junction). The 3′ splice site consists of three separate sequence elements: the branchpoint or branch site, a polypyrimidine tract and the 3′ consensus sequence (YAG). The branchpoint consensus sequence in mammals is YNYURAC (Y=pyrimidine). The underlined A is the site of branch formation. A polypyrimidine tract is located between the branchpoint and the splice site acceptor and is important for efficient branchpoint utilization and 3′ splice site recognition.

Further, PTMs comprising a 3′ acceptor site (AG) may be genetically engineered. Such PTMs may further comprise a pyrimidine tract and/or branchpoint sequence.

Recently, pre-messenger RNA introns beginning with the dinucleotide AU and ending with the dinucleotide AC have been identified and referred to as U12 introns. U12 intron sequences as well as any sequences that function as splice acceptor/donor sequences may also be used in PTMs.

A spacer region to separate the RNA splice site from the target binding domain is also included in the PTM. The spacer region can have features such as sequences that enhance trans-splicing to the target pre-mRNA. In a specific embodiment of the invention, initiation codon(s) and pre-mature termination codons may be incorporated into the PTMs of the invention as a mechanism for targeting selective degradation of unspliced RNAs thereby preventing translation and expression of unspliced RNAs from the nucleus into the cytoplasm. (see, Kim et al., 2001 Science 293:1832-1836)

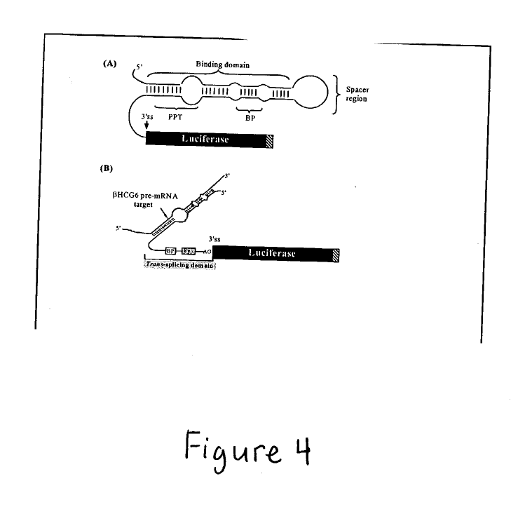

In a preferred embodiment of the invention, a “safety” is also incorporated into the spacer, binding domain, or elsewhere in the PTM to prevent non-specific trans-splicing. This is a region of the PTM that covers elements of the 3′ and/or 5′ splice site of the PTM by relatively weak complementarity, preventing non-specific trans-splicing. The PTM is designed in such a way that upon hybridization of the binding domain with a specific target pre-mRNA the 3′ and/or 5′ splice site is uncovered and becomes fully active. Schematic illustration of “safety mechanism is shown in FIG. 4.

The “safety” consists of one or more complementary stretches of cis-sequence (or could be a second, separate, strand of nucleic acid) which weakly binds to one or both sides of the PTM branchpoint, pyrimidine tract, 3′ splice site and/or 5′ splice site (splicing elements), or could bind to parts of the splicing elements themselves. This “safety” binding prevents the splicing elements from being active (i.e. block U2 snRNP or other splicing factors from attaching to the PTM splice site recognition elements). The binding of the “safety” may be disrupted by the binding of the target binding region of the PTM to the target pre-mRNA, thus exposing and activating the PTM splicing elements (making them available to trans-splice into the target pre-mRNA).

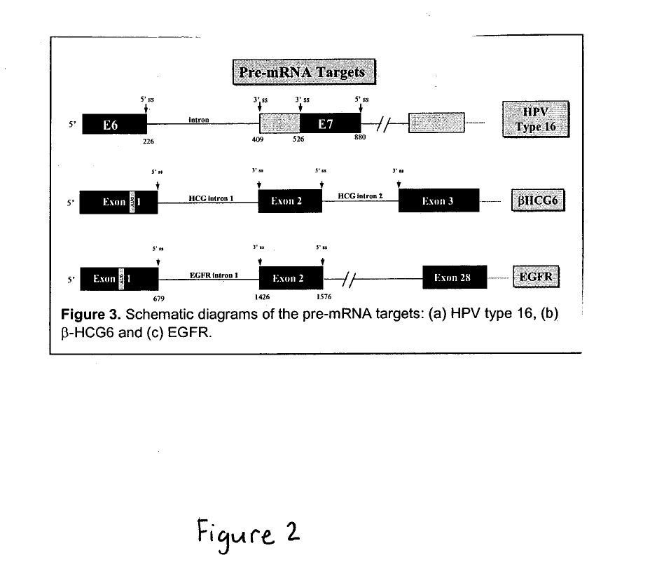

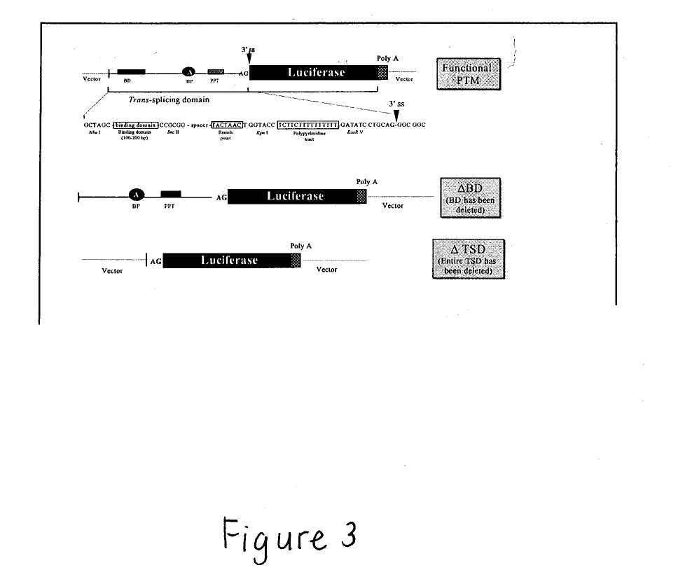

A nucleotide sequence encoding a translatable protein capable of producing a reporter molecule is included in the PTM of the invention. Such reporter genes include but are not limited to bioluminescent and fluorescent molecules, receptors, ion channel components, antibodies or fragments thereof, enzymes, and protein/peptide tags (Yu et al., 2000 Nature Medicine 6:933-937; MacLarent et al., 2000 Biol Psychiatry 48:337-348; Zaret et al., 2001 J. Nuclear Cardiology March/April 256-266; Ray et al., 2001 Seminars in Nuclear Medicine 31:312-320; Lok, 2001 Nature 412:372-374; Allport et al., 2001 Experimental Hematology 29:1237-1246; Berger and Gambhir, 2000 Breast Cancer Research 3:28-35; Cherry and Gambhir, 2001, ILAR Journal 42:219-232). Bioluminescent molecules include but are not limited to firefly, Renilla or bacterial luciferase. Fluorescent molecules include, for example, green fluorescent protein or red fluorescent protein. FIG. 3 is a representation of a prototype PTM designed to express a luciferase reporter molecule. FIG. 4 illustrates a PTM encoding luciferase including a safety mechanism.

In yet another embodiment of the invention, the reporter molecule may be an enzyme such as β-galactosidase (Louie et al., 2000 Nature Biotechnology 15:321-325), cytosine deaminase, herpes simplex virus type I thymidine kinase, creatine kinase (Yaghoubi et al., 2001 Human Imaging of Gene Expression 42:1225-1234; Yaghoubi et al., 2001 Gene Therapy 8:1072-1080; Iyer et al., 2001 J. Nuclear Medicine 42:96-105), or argininge kinase, to name a few. The enzyme is selected because of its ability to trap a specific radio labeled tracer by action of the enzyme on a chosen tracer.

Alternatively, the nucleotide sequences can encode for an intracellular and/or extracellular marker protein, such as a receptor, which is capable of binding to a labeled tracer that has a binding affinity for the expressed marker protein. Such proteins include, for example, the dopamine 2 receptor, somatostatin receptor, oxotechnetate-binding fusion proteins, gastrin-releasing peptide receptor, cathepsin D, the transferrin receptor or the CFTR C1 ion channel.

Nucleotide sequences encoding peptide tags, also referred to as epitope tags, may also be included in the structure of the PTMs of the invention. In a preferred embodiment of the invention, the epitope is one that is recognized by a specific antibody or binds to a specific ligand, each of which may be labeled, thereby providing a method for imaging of cells expressing the target pre-mRNA. Eptiopes that may be used include, but are not limited to, AU1, AU5, BTag, c-myc, FLAG, Glu-Glu, HA, His6, HSV, HTTPHH, IRS, KT3, Protien C, S-Tag, T7, V5, or VSV-G.

Additional features can be added to the PTM molecule either after, or before, the nucleotide sequence encoding a translatable protein, such as polyadenylation signals or 5′ splice sequences to enhance splicing, additional binding regions, “safety”-self complementary regions, additional splice sites, or protective groups to modulate the stability of the molecule and prevent degradation.

Additional features that may be incorporated into the PTMs of the invention include stop codons or other elements in the region between the binding domain and the splice site to prevent unspliced pre-mRNA expression. In another embodiment of the invention, PTMs can be generated with a second anti-sense binding domain downstream from the nucleotide sequences encoding a translatable protein to promote binding to the 3′ target intron or exon and to block the fixed authentic cis-5′ splice site (U5 and/or U1 binding sites).

PTMs may also be generated that require a double-trans-splicing reaction for generation of a chimeric trans-spliced product. PTMs designed to promote two trans-splicing reactions are engineered as described above, however, they contain both 5′ donor sites and 3′ splice acceptor sites. In addition, the PTMs may comprise two or more binding domains and spacer regions. The spacer regions may be placed between the multiple binding domains and splice sites or alternatively between the multiple binding domains.

Further elements such as a 3′ hairpin structure, circularized RNA, nucleotide base modification, or a synthetic analogs can be incorporated into synthetic PTMs to promote cell uptake or facilitate nuclear localization and spliceosomal recognition, and stability.

Additional features can be added to the PTM molecule such as polyadenylation signals, or enhancer sequences to enhance splicing, additional binding regions, “safety”-self complementary regions, additional splice sites, or protective groups to modulate the stability of the molecule and prevent degradation. In an embodiment of the invention, sequences referred to as exonic splicing enhancers may also be included in the structure of the synthetic PTMs. Transacting splicing factors, namely the serine/arginine-rich (SR) proteins, have been shown to interact with such exonic splicing enhancers and modulate splicing (See, Tacke et al., 1999, Curr. Opin. Cell Biol. 11:358-362; Tian et al., 2001, J. Biological Chemistry 276:33833-33839; Fu, 1995, RNA 1:663-680). Nuclear localization signals may also be included in the PTM molecule (Dingwell and Laskey, 1986, Ann. Rev. Cell Biol. 2:367-390; Dingwell and Laskey, 1991, Trends in Biochem. Sci. 16:478-481). Such nuclear localization signals can be used to enhance the transport of synthetic PTMs into the nucleus where trans-splicing occurs. In addition, sequences may be used that enhance the retention of un-spliced PTMs in the nucleus (Boelans et al., 1995 RNA 1:273-83; Good et al. 1997 Gene Ther. 4:45-54)

Additionally, when engineering PTMs for use in plant cells it may not be necessary to include branchpoint sequences or polypyrimidine tracts as these sequences generally are not utilized for intron processing in plants. However, a 3′ splice acceptor site and/or 5′ splice donor site, such as those required for splicing in vertebrates and yeast, will be included. Further, the efficiency of splicing in plants may be increased by including UA-rich intronic sequences. The skilled artisan will recognize that any sequences that are capable of mediating a trans-splicing reaction in plants may be used.

When using the synthetic PTMs, the PTMs of the invention can be modified at the base moiety, sugar moiety, or phosphate backbone, for example, to improve stability of the molecule, hybridization to the target mRNA, transport into the cell, etc. For example, modification of a PTM to reduce the overall charge can enhance the cellular uptake of the molecule. In addition modifications can be made to reduce susceptibility to nuclease or chemical degradation. The nucleic acid molecules may be synthesized in such a way as to be conjugated to another molecule such as a peptides (e.g., for targeting host cell receptors in vivo), or an agent facilitating transport across the cell membrane (see, e.g., Letsinger et al., 1989, Proc. Natl. Acad. Sci. USA 86:6553-6556; Lemaitre et al., 1987, Proc. Natl. Acad. Sci. 84:648-652; PCT Publication No. W088/09810, published Dec. 15, 1988) or the blood-brain barrier (see, e.g., PCT Publication No. W089/10134, published Apr. 25, 1988), hybridization-triggered cleavage agents (see, e.g., Krol et al., 1988, BioTechniques 6:958-976) or intercalating agents (see, e.g., Zon, 1988, Pharm. Res. 5:539-549). To this end, the nucleic acid molecules may be conjugated to another molecule, e.g., a peptide, hybridization triggered cross-linking agent, transport agent, hybridization-triggered cleavage agent, etc.

Various other well-known modifications to the nucleic acid molecules can be introduced as a means of increasing stability and half-life. Possible modifications include, but are not limited to, the addition of flanking sequences of ribonucleotides to the 5′ and/or 3′ ends of the molecule. In some circumstances where increased stability is desired, nucleic acids having modified intemucleoside linkages such as 2′-0-methylation may be preferred. Nucleic acids containing modified intemucleoside linkages may be synthesized using reagents and methods that are well known in the art (see, Uhlmann et al., 1990, Chem. Rev. 90:543-584; Schneider et al., 1990, Tetrahedron Lett. 31:335 and references cited therein).

Synthetic PTMs of the present invention are preferably modified in such a way as to increase their stability. Since RNA molecules are sensitive to cleavage by cellular ribonucleases, it may be preferable to use as the competitive inhibitor a chemically modified oligonucleotide (or combination of oligonucleotides) that mimics the action of the RNA binding sequence but is less sensitive to nuclease degradation. In addition, the synthetic PTMs can be produced as nuclease resistant circular molecules with enhanced stability (Puttaraju et al., 1995, Nucleic Acids Symposium Series No. 33:49-51; Puttaraju et al., 1993, Nucleic Acid Research 21:4253-4258). Other modifications may also be required, for example to enhance binding, to enhance cellular uptake, to improve pharmacology or pharmacokinetics or to improve other pharmaceutically desirable characteristics.

Modifications, which may be made to the structure of the synthetic PTMs include but are not limited to backbone modifications such as use of: (i) phosphorothioates (X or Y or W or Z=S or any combination of two or more with the remainder as O). e.g. Y═S (Stein, C. A., et al., 1988, Nucleic Acids Res., 16:3209-3221), X═S (Cosstick, R., et al., 1989, Tetrahedron Letters, 30, 4693-4696), Y and Z═S (Brill, W. K.-D., et al., 1989, J. Amer. Chem. Soc., 111:2321-2322); (ii) methylphosphonates (e.g. Z=methyl (Miller, P. S., et al., 1980, J. Biol. Chem., 255:9659-9665); (iii) phosphoramidates (Z=N-(alkyl)2 e.g. alkyl methyl, ethyl, butyl) (Z=morpholine or piperazine) (Agrawal, S., et al., 1988, Proc. Natl. Acad. Sci. USA 85:7079-7083) (X or W═NH) (Mag, M., et al., 1988, Nucleic Acids Res., 16:3525-3543); (iv) phosphotriesters (Z═O-alkyl e.g. methyl, ethyl, etc) (Miller, P. S., et al., 1982, Biochemistry, 21:5468-5474); and (v) phosphorus-free linkages (e.g. carbamate, acetamidate, acetate) (Gait, M. J., et al., 1974, J. Chem. Soc. Perkin I, 1684-1686; Gait, M. J., et al., 1979, J. Chem. Soc. Perkin I, 1389-1394). See also, Sazani et al., 1974, Nucleic Acids Research 29:3965-3974.

In addition, sugar modifications may be incorporated into the PTMs of the invention. Such modifications include but are not limited to the use of: (i) 2′-ribonucleosides (R═H); (ii) 2′-O-methylated nucleosides (R═OMe) (Sproat, B. S., et al., 1989, Nucleic Acids Res., 17:3373-3386); and (iii) 2′-fluoro-2′-ribonucleosides (R═F) (Krug, A., et al., 1989, Nucleosides and Nucleotides, 8:1473-1483).

Further, base modifications that may be made to the PTMs, including but not limited to use of: (i) pyrimidine derivatives substituted in the 5-position (e.g. methyl, bromo, fluoro etc) or replacing a carbonyl group by an amino group (Piccirilli, J. A., et al., 1990, Nature, 343:33-37); (ii) purine derivatives lacking specific nitrogen atoms (e.g. 7-deaza adenine, hypoxanthine) or functionalized in the 8-position (e.g. 8-azido adenine, 8-bromo adenine) (for a review see Jones, A. S., 1979, Int. J. Biolog. Macromolecules, 1: 194-207).

In addition, the PTMs may be covalently linked to reactive functional groups, such as: (i) psoralens (Miller, P. S., et al., 1988, Nucleic Acids Res., Special Pub. No. 20, 113-114), phenanthrolines (Sun, J-S., et al., 1988, Biochemistry, 27:6039-6045), mustards (Vlassov, V. V., et al., 1988, Gene, 72:313-322) (irreversible cross-linking agents with or without the need for co-reagents); (ii) acridine (intercalating agents) (Helene, C., et al., 1985, Biochimie, 67:777-783); (iii) thiol derivatives (reversible disulphide formation with proteins) (Connolly, B. A., and Newman, P. C., 1989, Nucleic Acids Res., 17:4957-4974); (iv) aldehydes (Schiffs base formation); (v) azido, bromo groups (UV cross-linking); or (vi) ellipticines (photolytic cross-linking) (Perrouault, L., et al., 1990, Nature, 344:358-360).

In an embodiment of the invention, oligonucleotide mimetics in which the sugar and internucleoside linkage, i.e., the backbone of the nucleotide units, are replaced with novel groups can be used. For example, one such oligonucleotide mimetic which has been shown to bind with a higher affinity to DNA and RNA than natural oligonucleotides is referred to as a peptide nucleic acid (PNA) (for review see, Uhlmann, E. 1998, Biol. Chem. 379:1045-52). Thus, PNA may be incorporated into synthetic PTMs to increase their stability and/or binding affinity for the target pre-mRNA.

In another embodiment of the invention synthetic PTMs may covalently linked to lipophilic groups or other reagents capable of improving uptake by cells. For example, the PTM molecules may be covalently linked to: (i) cholesterol (Letsinger, R. L., et al., 1989, Proc. Natl. Acad. Sci. USA, 86:6553-6556); (ii) polyamines (Lemaitre, M., et al., 1987, Proc. Natl. Acad. Sci, USA, 84:648-652); other soluble polymers (e.g. polyethylene glycol) to improve the efficiently with which the PTMs are delivered to a cell. In addition, combinations of the above identified modifications may be utilized to increase the stability and delivery of PTMs into the target cell.

The PTMs of the invention can be used in diagnostic methods designed to identify cells expressing a gene, which may be associated with a disorder or disease. The methods of the present invention comprise delivering to the target cell a PTM which may be in any form used by one skilled in the art, for example, a synthetic RNA molecule, or a DNA vector which is transcribed into a RNA molecule, wherein said PTM binds to the target pre-mRNA and mediates a trans-splicing reaction resulting in formation of a chimeric RNA comprising the portion of the PTM molecule encoding a reporter molecule trans-spliced to a portion of the pre-mRNA.

Synthesis of the Trans-Splicing Molecules

The nucleic acid molecules of the invention can be RNA or DNA or derivatives or modified versions thereof, single-stranded or double-stranded. By nucleic acid is meant a PTM molecule or a nucleic acid molecule encoding a PTM molecule, whether composed of deoxyribonucleotides or ribonucleotides, and whether composed of phosphodiester linkages or modified linkages. The term nucleic acid also specifically includes nucleic acids composed of bases other than the five biologically occurring bases (adenine, guanine, thymine, cytosine and uracil).

The RNA and DNA molecules of the invention can be prepared by any method known in the art for the synthesis of DNA and RNA molecules. For example, the nucleic acids may be chemically synthesized using commercially available reagents and synthesizers by methods that are well known in the art (Gait, 1985, Oligonucleotide Synthesis: A Practical Approach, IRL Press, Oxford, England). Alternatively, RNA molecules can be generated by in vitro and in vivo transcription of DNA sequences encoding the RNA molecule. Such DNA sequences can be incorporated into a wide variety of vectors which incorporate suitable RNA polymerase promoters such as the T7 or SP6 polymerase. RNAs may be produced in high yield via in vitro transcription using plasmids such as SPS65. (Promega Corporation, Madison, Wis.). In addition, RNA amplification methods such as Q-β amplification can be utilized to produce RNAs.

The nucleic acid molecules can be modified at the base moiety, sugar moiety, or phosphate backbone, for example, to improve stability of the molecule, hybridization, transport into the cell, etc. For example, modification of a PTM to reduce the overall charge can enhance the cellular uptake of the molecule. In addition modifications can be made to reduce susceptibility to nuclease degradation. The nucleic acid molecules may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. W088/09810, published Dec. 15, 1988) or the blood-brain barrier (see, e.g., PCT Publication No. W089/10134, published Apr. 25, 1988), hybridization-triggered cleavage agents. (See, e.g., Krol et al., 1988, BioTechniques 6:958-976) or intercalating agents. (See, e.g., Zon, 1988, Pharm. Res. 5:539-549). To this end, the nucleic acid molecules may be conjugated to another molecule, e.g., a peptide, hybridization triggered cross-linking agent, transport agent, hybridization-triggered cleavage agent, etc. Various other well-known modifications to the nucleic acid molecules can be introduced as a means of increasing intracellular stability and half-life. Possible modifications include, but are not limited to, the addition of flanking sequences of ribo- or deoxy- nucleotides to the 5′ and/or 3′ ends of the molecule. In some circumstances where increased stability is desired, nucleic acids having modified internucleoside linkages such as 2′-0-methylation may be preferred. Nucleic acids containing modified internucleoside linkages may be synthesized using reagents and methods that are well known in the art (see, Uhlmann et al., 1990, Chem. Rev. 90:543-584; Schneider et al., 1990, Tetrahedron Lett. 31:335 and references sited therein).

The nucleic acids may be purified by any suitable means, as are well known in the art. For example, the nucleic acids can be purified by reverse phase chromatography or gel electrophoresis. Of course, the skilled artisan will recognize that the method of purification will depend in part on the size and charge of the nucleic acid to be purified.

In instances where a nucleic acid molecule encoding a PTM is utilized, cloning techniques known in the art may be used for cloning of the nucleic acid molecule into an expression vector. Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

The DNA encoding the PTM of interest may be recombinantly engineered into a variety of host vector systems that also provide for replication of the DNA in large scale and contain the necessary elements for directing the transcription of the PTM. The use of such a construct to transfect target cells in the patient will result in the transcription of sufficient amounts of PTMs that will form complementary base pairs with the endogenously expressed pre-mRNA targets and thereby facilitate a trans-splicing reaction between the complexed nucleic acid molecules. For example, a vector can be introduced in vivo such that it is taken up by a cell and directs the transcription of the PTM molecule. Such a vector can remain episomal or become chromosomally integrated, as long as it can be transcribed to produce the desired RNA. Such vectors can be constructed by recombinant DNA technology methods standard in the art.

Vectors encoding the PTM of interest can be plasmid, viral, or others known in the art, used for replication and expression in mammalian cells. Expression of the sequence encoding the PTM can be regulated by any promoter known in the art to act in mammalian, preferably human cells. Such promoters can be inducible or constitutive. Such promoters include but are not limited to: the SV40 early promoter region (Benoist, C. and Chambon, P. 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), the viral CMV promoter, the human chorionic gonadotropin-β promoter (Hollenberg et al., 1994, Mol. Cell. Endocrinology 106:111-119), etc. Any type of plasmid, cosmid, YAC or viral vector can be used to prepare the recombinant DNA construct which can be introduced directly into the tissue site. Alternatively, viral vectors can be used which selectively infect the desired target cell.

PTM Mediated Imaging of Gene Expression

The present invention provides methods and compositions for imaging of gene expression in cells. The compositions and methods of the invention can be used to diagnose cancer, viral, bacterial, parasitic, or fungal infections, autoimmune disorders, and other pathological conditions in which the condition is associated with expression of a specific mRNA. The invention also provides screening assays for identifying agents capable of modulating gene expression and assays for identifying protein/protein, DNA/protein and RNA/protein interactions within a cell. The methods and compositions of the invention may additionally be used as a detection system designed to indicate when an organism or cell has been exposed to a specific compound, such as a toxic or noxious compound.

The diagnostic methods of the invention comprise contacting a test subject, or a sample derived from a test subject, with a PTM or a nucleic acid molecule encoding a PTM. If the target pre-mRNA is expressed in the sample, or in the test subject, a trans-splicing reaction will occur resulting in the production of a chimeric RNA molecule capable of encoding a reporter molecule. Detection of the reporter molecule indicates the presence of the substance, disorder or disease.

Various delivery systems are known and can be used to transfer the compositions of the invention into cells, e.g. encapsulation in liposomes, microparticles, microcapsules, recombinant cells capable of expressing the composition, receptor-mediated endocytosis (see, e.g., Wu and Wu, 1987, J. Biol. Chem. 262:4429-4432), construction of a nucleic acid as part of a retroviral or other vector, injection of DNA, electroporation, calcium phosphate mediated transfection, etc.

Any of the methods for gene delivery into a host cell available in the art can be used according to the present invention. For general reviews of the methods of gene delivery see Strauss, M. and Barranger, J. A., 1997, Concepts in Gene Therapy, by Walter de Gruyter & Co., Berlin; Goldspiel et al., 1993, Clinical Pharmacy 12:488-505; Wu and Wu, 1991, Biotherapy 3:87-95; Tolstoshev, 1993, Ann. Rev. Pharmacol. Toxicol. 33:573-596; Mulligan, 1993, Science 260:926-932; and Morgan and Anderson, 1993, Ann. Rev. Biochem. 62:191-217; 1993, TIBTECH 11(5):155-215. Exemplary methods are described below.

In a specific embodiment, the nucleic acid is directly administered in vivo, where it is expressed to produce the PTM. This can be accomplished by any of numerous methods known in the art, e.g., by constructing it as part of an appropriate nucleic acid expression vector and administering it so that it becomes intracellular, e.g. by infection using a defective or attenuated retroviral or other viral vector (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering it in linkage to a peptide which is known to enter the nucleus, by administering it in linkage to a ligand subject to receptor-mediated endocytosis (see e.g., Wu and Wu, 1987, J. Biol. Chem. 262:4429-4432).

In a specific embodiment, a viral vector that contains the PTM can be used. For example, a retroviral vector can be utilized that has been modified to delete retroviral sequences that are not necessary for packaging of the viral genome and integration into host cell DNA (see Miller et al., 1993, Meth. Enzymol. 217:581-599). Alternatively, adenoviral or adeno-associated viral vectors can be used for gene delivery to cells or tissues. (See, Kozarsky and Wilson, 1993, Current Opinion in Genetics and Development 3:499-503 for a review of adenovirus-based gene delivery).

Another approach to PTM delivery into a cell involves transferring the PTM to cells in tissue culture by such methods as electroporation, lipofection, calcium phosphate mediated transfection, or viral infection.

The present invention also provides for compositions comprising an effective amount of a PTM or a nucleic acid encoding a PTM, and an acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the PTM is administered. Examples of suitable pharmaceutical carriers are described in “Remington's Pharmaceutical sciences” by E. W. Martin.

Once the PTM molecule has been contacted with the test sample, or subject, cells will be imaged or assayed to detect expression of the reporter molecule. In instances where the reporter molecule does not provide a label for imaging, an enzymatic substrate or tracer molecule is added to detect expression of the reporter molecule. The tracer molecule can be labeled in a variety of different ways, including but not limited to, fluorescent, bioluminescent and radioactive labeling. The tracer is designed to bind to the reporter molecule thereby providing a signal for cells expressing the target pre-mRNA.

Cells can be imaged using a number of methods well known to those of skill in the art. Such methods include, for example, use of a CCD low-light monitoring system, positron emission tomography (PET), single photon emission computed tomography (SPECT), magnetic resonance imaging (MRI), ultrasound (US), and endoscopic optical coherence tomography.

In addition to diagnostic uses, the present invention may be utilized to detect specific gene expression in plants, such as for example, crop plants. Such specific gene expression may include the expression of RNAs encoded by plant pathogens or genes activated in the plant in response to infection or damage.

The methods and compositions of the invention may also be used in detection systems designed to indicate whether an organism has been exposed to a specific compound. Such compounds include, for example, toxic and/or noxious compounds. In such an embodiment of the invention, the PTM is designed to include a target binding domain complementary to a specific target pre-mRNA the expression of which is induced by exposure to the compound.

In yet another embodiment of the invention, a screening assay is provided for identification of agents capable of modulating gene expression. The assays of the invention comprise (i) contacting a cell containing a PTM that targets a pre-mRNA of interest and is capable of expressing a reporter molecule with a test agent; (ii) measuring the level of reporter molecule expressed within the cell and comparing that level to the level obtained in the absence of a test agent; wherein a difference in the amount of reporter molecule in the presence of the test agent versus absence of the test agent indicates the identification of a agent capable of modulating gene expression.

### EXAMPLE

Trans-Splicing of Luciferase Into Exogenously Expressed Genes

The following example describes the production of PTMs designed to encode a reporter molecule.

Materials and Methods

Design and Construction of PTMs

The binding domain of PTMs is assembled from either PCR products or annealed oligonucleotides. The coding sequence for firefly luciferase is generated by PCR using commercially available plasmid cDNA (Promega). To reduce the possibility of self-expression of the PTM prior to trans-splicing, the initiator AUG codon may be eliminated from the coding sequence during PCR amplification. As an example Luc-PTM1, shown in FIG. 3, consists of an antisense target binding domain of 100-200 nt complementary to β-HCG6 intron 1, a spacer sequence, a yeast branchpoint consensus sequence (UACUAAC), an extensive polypyrimidine tract (12-15 pyrimidines), a 3′ acceptor site (AG dinucleotide) followed by the complete coding for firefly luciferase minus the initiator codon. Unique restriction sites are placed between each of the PTM elements, facilitating the replacement of individual elements. In addition, the binding domain may contain alternate sites that initiate transcription out of frame from the reporter gene thereby preventing translation and expression of unspliced PTMs.

Optimization of PTMs. A number of approaches can be taken to improve the characteristics of luciferase PTMs as described below.

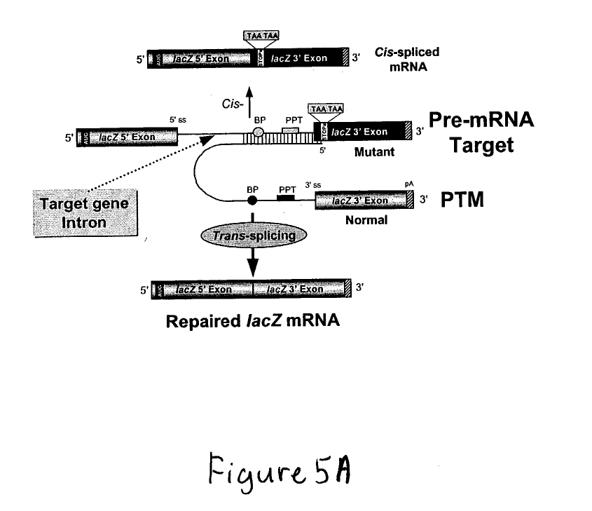

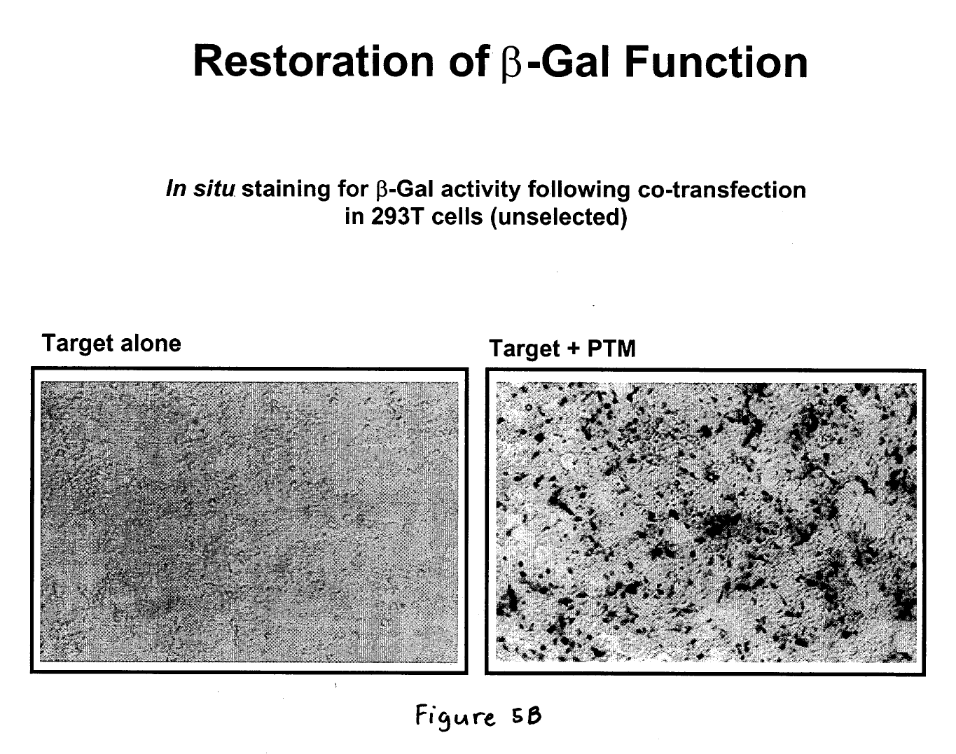

Binding domain: Several different forms of binding domain can be utilized. Using lacZ as a pre-screening model (FIG. 5) it was demonstrated that some PTMs with longer binding domains trans-spliced with higher frequency to the intended target pre-mRNA compared to PTMs with shorter binding domain (Puttaraju et al., 2001). This data suggest that longer binding domains may increase the interaction of the PTM with the target. The increased interaction between the target and PTM can enhance both the efficiency and specificity of trans-splicing reaction.

Initially, PTMs with binding domains spanning 100-200 nucleotides are constructed and assayed. Safety PTMs with stem loop binding domains may also be produced. Based on the efficiency of the trans-splicing reactions, if necessary, binding domains longer than this (200-400 nt) can be utilized. Binding domains can also be designed to target different regions of the same intron, e.g. binding domains close to the donor vs. the acceptor site, or binding domains targeted to completely different introns.

Screening for PTM cis-splicing: To reduce the possibility of cis-splicing in the trans-splicing domain (TSD) of the PTM prior to target binding, TSD sequences are analyzed for the presence of potential 5′ and 3′ cryptic splice sites (GU-AG and AT-AC, U12 type introns) prior to construction of the binding domain. This is especially important for the linear binding domain PTMs (see below) because their intended splice sites may be available for binding splicing factors at all times. For each situation a single site in the TSD that could potentially be used as a 3′ cryptic splice site is usually altered from TAG/G to TTGC. The PTM can be screened by RT-PCR to check for the presence of major products (cis or trans) of unexpected size. PTM coding sequences may also be screened and altered if necessary in a similar manner.

3′ splice elements. 3′ splice elements including the branchpoint (BP), the polypyrimidine tract (PPT) and a 3′ acceptor site (AG dinucleotide) may also be included. Trans-splicing can be modulated by changing the sequence of the BP and the length and composition of the PPT. A yeast consensus branchpoint sequence UACUAAC provides a greater rate of trans-splicing in mammalian cells (Puttaraju et al., 1999).

Modulating specificity with “safety” stems. Initial experiments can be performed with “linear” PTMs to maximize the trans-splicing efficiency. Linear PTMs have a binding domain designed to exist in a single stranded configuration to maximize base pairing to target and trans-splicing efficiency. To achieve a higher degree of targeting specificity and trans-splicing, the trans-splicing domain is designed to include intra-molecular stems (termed ‘safety PTM’) designed to mask the 3′ splicing elements carried in the PTM from spliceosomal components prior to target binding. Base pairing between free portions of the PTM binding domain with the target is thought to facilitate the unwinding of the safety stem, allowing the splicing factors access to bind to the splice site and initiate trans-splicing. A schematic drawing of the safety mechanism is illustrated in FIG. 4. An array of safety PTM designs are constructed and tested by varying the strength of the safety stem and assessing trans-splicing efficiency and specificity. For example, a safety PTM targeting the CFTR pre-mRNA has been designed with equivalent efficiency in trans-splicing as its parental PTM with improved specificity (Mansfield et al, 2000).

Untranslated regions. Modification of 3′ UTR and RNA processing signals are also carried out to increase RNA processing and stability. To increase the stability of trans-spliced messages and ultimately the level of luciferase activity, alternative polyadenylation signals may be engineered in the 3′ untranslated sequence. To maximize the efficiency of 3′ end cleavage and polyadenylation of trans-spliced mRNA, each PTM construct can be modified by including GT rich sequences (consensus YGTGTTYY) downstream of the poly-A signal. This consensus, initially identified in herpes simplex virus genes, has been shown to be present in a large number of mammalian genes. Other modifications are also possible.

Cell Models

The PTM modifications described above are tested in the following cell based models. HPV infected/expressing cell lines including CaSki and SiHa cells are cervical cancer cell lines that express high and low levels of HPV RNA, respectively. β-HCG6 cell lines include H1299 which is a lung adenocarcinoma cell line that expresses low levels of target transcript. JEG-3 is a coriocarcinoma cell line that expresses considerably higher levels of β-HCG6 mRNAs. EGFR expressing cell lines include A431, an epidermoid carcinoma cell line that overexpresses EGFR and MCF7; an epithelial breast cancer cell line is used extensively in cancer research. In addition, Eccles et al., has published on a variety of tumor cell lines that have expressed varying levels of EGFR (O-Charoenrat et al., 2000).

Assaying for Trans-Splicing: Targeting Endogenous Transcripts

Cells are transfected with PTM plasmids using Lipofectamine or TransFast reagents. Trans-splicing efficiency and specificity is assessed by performing luciferase activity assays and RT-PCR analysis of cells (transiently transfected or neomycin selected populations).

Luciferase activity assays. Trans-splicing mediated luciferase activity is initially monitored in cell extracts using luciferase assay reagents (Promega). If necessary, dual reporters are used as a means to measure the specificity of trans-splicing. This approach provides an internal control that is useful to account for the experimental variations caused by differences in cell viability, transfection efficiency, and cell lysis efficiency. The studies are performed with luciferase based PTMs including, for example, firefly and Renilla luciferases. Each marker has distinct kinetics and emission spectra, dissimilar structure and different substrate requirements, properties that make it possible to selectively discriminate between their respective bioluminescent reactions. Controls are performed to exclude the possibility that chimeric products between luciferase and targets are not being generated by recombination events.

Transfected cells are imaged using a CCD low-light monitoring system. In addition, trans-splicing efficiency at the RNA level is determined by real time quantitative RT-PCR analysis of total RNA samples using target and PTM specific primers.

It may be more efficient to initially select the best PTM candidates for the pre-mRNA targets, using cell lines that express the target RNA from a stably integrated mini-gene construct. The advantages of this system include the following: (i) the cell lines express target RNA from a genomic locus recapitulating the endogenous system, (ii) the cells are easy to transfect, and (iii) high levels of target transcript is produced, making it quicker and easier to assess differences in efficiency and specificity between PTMs. Cell lines that express different levels of the target pre-mRNA or use inducible promoters to modulate expression level may also be used. Inducible promoters will facilitate the determination of sensitivity of trans-splicing and correlation of target mRNA concentration to luciferase signal.

A simple pre-screening model based on the β-galactosidase repair model (Puttaraju et al., 2001) (FIG. 5A) can also be utilized. This system involves the insertion of the target introns from β-HCG6, HPV or EGFR into a mutant luciferase gene. The target is established in a stable cell line or cotransfected with PTMs. Efficiency will be quickly assessed by RT-PCR and luciferase activity assays. This type of system has proved extremely useful as a pre-screen for PTM binding domain sequences (Puttaraju et al., 2001).

### EXAMPLE

Luciferase Model for Trans-Splicing

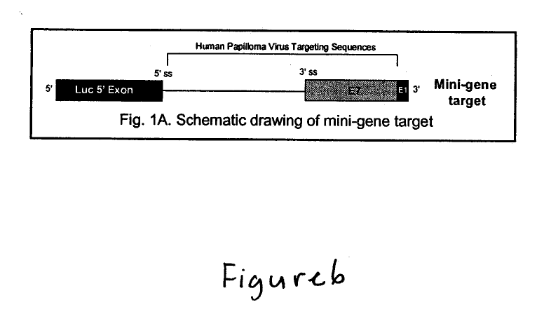

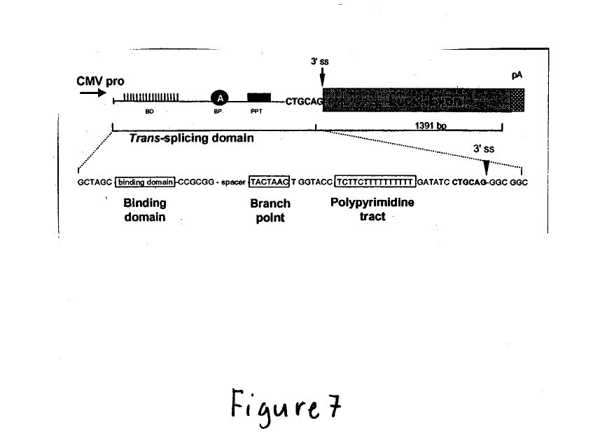

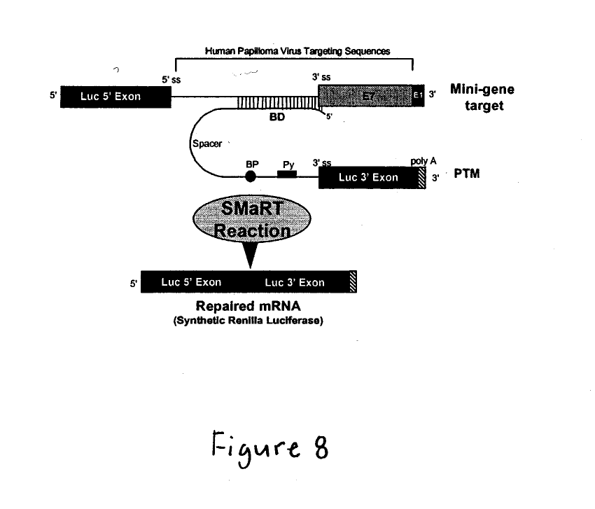

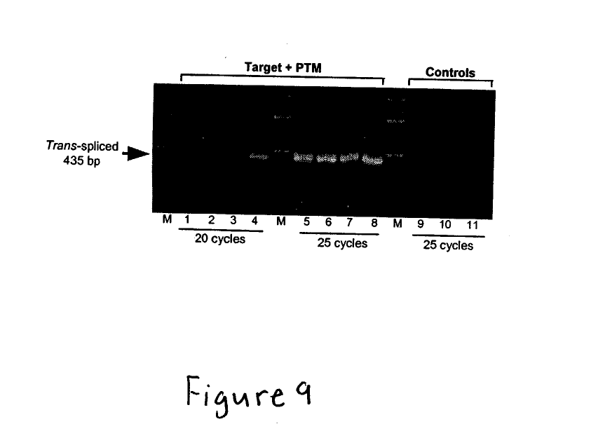

To evaluate the potential use of spliceosome mediated RNA trans-splicing for molecular imaging of gene expression in real time a screening luciferase model was developed. To quantify the level of luciferase generated by trans-splicing in cells and small animal models, a pre-mRNA target was constructed that expressed part of the synthetic Renilla luciferase sequence, coupled to the coding sequences for HPV E7 and the sequence of HPV immediate upstream of E7 from the human papilloma virus (HPV) (FIG. 6). The chimeric pre-mRNA target undergoes normal cis-splicing to produce an mRNA but no luciferase activity. A pre-trans-splicing molecule (PTM) was engineered that should base pair with the target intron and trans-splice the 3′ luciferase ‘exon’, into the target producing full length luciferase mRNA capable of producing luciferase activity (FIGS. 7 and 8). This PTM (Luc-PTM13) contains an 80 bp targeting domain that is complementary to intron 1 of HPV mRNA, a branchpoint (UACUAAC) and polypyrimidine tract, AG dinucleotide acceptor followed by 3′ hemi luciferase ‘exon’. This region was selected based on the results targeting this clinically relevant splice site in HPV mRNA, where as high as 70% trans-splicing efficiency was achieved in cell culture models. A splice mutant was also constructed by deleting both the branchpoint and polypyrimidine sequences. Using these constructs, accurate trans-splicing of luciferase PTM13 (Luc-PTM13) into HPV-LucT1 target in human cells was demonstrated. Human embryonic kidney cells were transfected with either target, PTM alone as controls or co-transfected with both target and PTM expression plasmids. In a separate transfection target and splice mutant PTM were co-transfected. RT-PCR analysis of total RNA using target and PTM specific primers produced the expected trans-spliced (435 bp) product only in cells that contained both target and PTM but not in controls (target, PTM alone and target+splice mutant PTM) (FIG. 9).

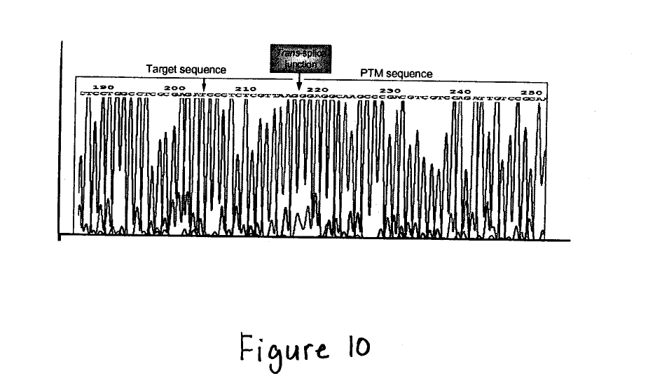

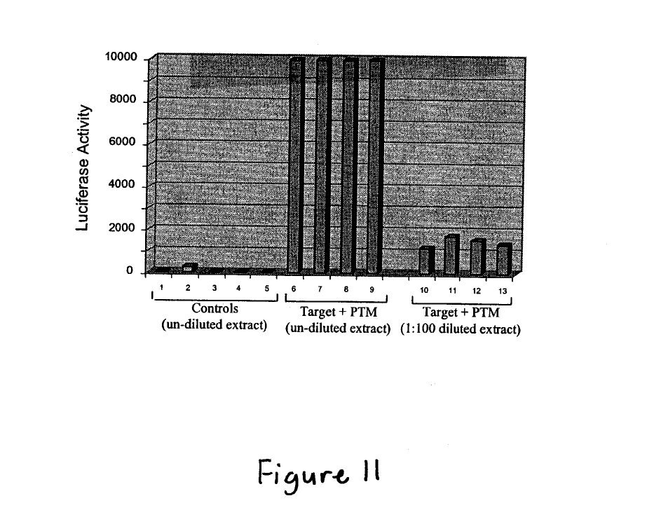

Direct sequence of this RT-PCR product confirmed the accurate trans-splicing between the target and PTM (FIG. 10). The efficiency of trans-splicing mediated restoration of function was confirmed at the protein level by assaying for luciferase activity. The results are summarized in FIG. 11. Co-transfection of a specific target with Luc-PTM13 resulted in the repair and restoration of luciferase function that is on the order of 4-logs over the background. No luciferase activity above background was detected in controls or with splice mutant PTM suggesting that the restoration of luciferase function is due to trans-splicing (FIG. 11).

In a parallel study, PTMs that trans-splice complete luciferase coding (minus the 1st ATG codon) into the β-HCG6 pre-mRNA target were constructed. Preliminary results suggest that these PTMs are self-expressing. This was not overly surprising because these PTMs may be using one of the internal methionines contained in the coding sequence of luciferase for translation. To circumvent this the following approaches may be taken: (1) conversion of the methionines at amino acid position 8 and 27, for example, of luciferase coding sequence to isolucine; (2) adding a nuclear retention signal (U6 snRNA) at the 5′ end to prevent PTM export prior to trans-splicing, and (3) designing PTMs such that they would initiate translation out-of-frame if the PTMs are exported into the cytoplasm without undergoing trans-splicing.

### EXAMPLE 8

Imaging Gene Expression in Cells Using Synthetic PTM RNA

Material and Methods

In Vitro Transcription and Purification of RNA

Template DNA: Plasmids, pc3.1Luc-PTM13, pc3.1Luc-PTM14 and pc3.1Luc-13-BP/PPT (splice mutant PTM) containing T7 promoter were digested with Hind III restriction enzyme at 37° C. The products were extracted with buffered phenol followed by chloroform or purified using Qiaquick PCR purification kit (Qiagen). The DNA was recovered by ethanol precipitation and washed twice with 70% ethanol, air dried for 5 minutes, re-suspended with sterile water and used for in vitro transcription.

In vitro transcription: In vitro transcription was performed in 20 μl reaction using mMESSAGE mMACHINE high yield capped RNA transcription kit for capped RNA following manufacturers protocol (Ambion) and 1 μg of linearized plasmid DNA template. The reactions were incubated at 37° C. for 2-3 hours and the DNA template was destroyed by adding 1 μl of DNase 1 (2U/μl) and continuing the incubation at 37° C. for an additional 45 minutes. The poly A tail (˜150-200 nt) was added to the in vitro transcribed RNA using poly A tailing kit (Ambion) by incubating the reaction with E. coli poly A polymerase and ATP by incubating at 37° C. for 60 minutes. Reactions were terminated by placing the tubes on ice and the RNAs were purified as described below.

RNA Purification: In vitro transcribed, poly A tailed RNA was purified using MEGAclear purification kit (Ambion) which is designed to remove unincorporated free nucleotides, short oligonucleotides, proteins and salts from RNA. Briefly, RNA was bound to the filter cartridge, washed with washing buffer and eluted with a low salt buffer.

Synthetic RNA Transfections

The day before transfection, 1×10⁶ 293T cells were plated in 60 mm tissue culture plate with 5 ml of DMEM growth medium supplemented with 10% FBS. Cells were incubated at 37° C. in a CO₂ incubator for 12-14 hours or until the cells are 70-80% confluent. Before transfection, the cells were washed with 2 ml Opti-MEM 1 reduced serum medium. The RNA-Lipid complexes were prepared by adding 1.7 ml of Opti-MEM 1 into 2 ml tube followed by 8 μl of DMRIE-C transfection reagent (Invitrogen) and mixed briefly. To the above mix, known amount of the in vitro transcribed, poly A tailed and purified RNA was added, vortexed briefly and immediately added drop wise on to the cells. The cells were incubated for 4 hours at 37° C. and then the transfection medium was replaced with complete growth medium (DMEM with 10% FBS). After incubating for an additional 24-48 hours, the plates were rinsed with PBS once, cells harvested and total RNA was isolated using MasterPure RNA purification kit (Epicenter Technologies, Madison, Wis.). Contaminating DNA in the RNA preparation was removed by treating with DNase 1 at 37° C. for 30-45 minutes and the product RNA was purified as recommended in the kit.

Reverse Transcription and Polymerase-Chain Reaction (RT-PCR)

Total RNA (2.5 μg) from the transfections was converted to cDNA using the MMLV reverse transcriptase enzyme (Promega) in a 25 μl reaction following the manufacturers protocol with the addition of 50 units RNase Inhibitor (Invitrogen) and 200 ng Luc-11R PTM specific primer

(5′AAGCTTTTACTGCTCGTTCTTCAGCACGC). cDNA synthesis reactions were incubated at 42° C. for 60 minutes followed by incubation at 95° C. for 5 minutes. This cDNA template was used for PCR reactions. PCR amplifications were performed using 100 ng of primers and 1 μl template (RT reaction) per 50 μl PCR reaction. A typical reaction contained ˜25 ng of cDNA template, 100 ng of primers:

Luc-33R (5′-CAGGGTCGGACTCGATGAAC) and,

Luc-34F, 5′-GGATATCGCCCTGATCAAGAG) 1×REDTaq PCR buffer (10 mM Tris-HCl, pH 8.3, 50 mM KCl, 1.1 mM MnCl₂ and 0.1% gelatin), 200 μM dNTPs and 1.5 units of REDTaq DNA polymerase (Sigma, Saint Louis, Mo.). PCR reactions were performed with an initial pre-heating at 94° C. for 2 minutes 30 seconds followed by 25-30 cycles of 94° C. for 30 seconds (denaturation), 60° C. for 36 seconds (annealing) and 72° C. for 1 minute (extension) followed by a final extension at 72° C. for 7 minutes. The PCR products were then analyzed on a 2% agarose gel and the DNA bands were visualized by staining with ethidium bromide.

Assay for Renilla Luciferase Activity

48 hours post-transfection the cells were rinsed once with 1× phosphate buffered saline (PBS) and harvested following the standard procedures. The cell pellet was re-suspending in 100 μl of lysis buffer, lysed and Renilla Luciferase activity was measured by the Renilla Luciferase assay system (Promega, Madison, Wis., USA) using a Turner 20/20 TD luminometer.

Results

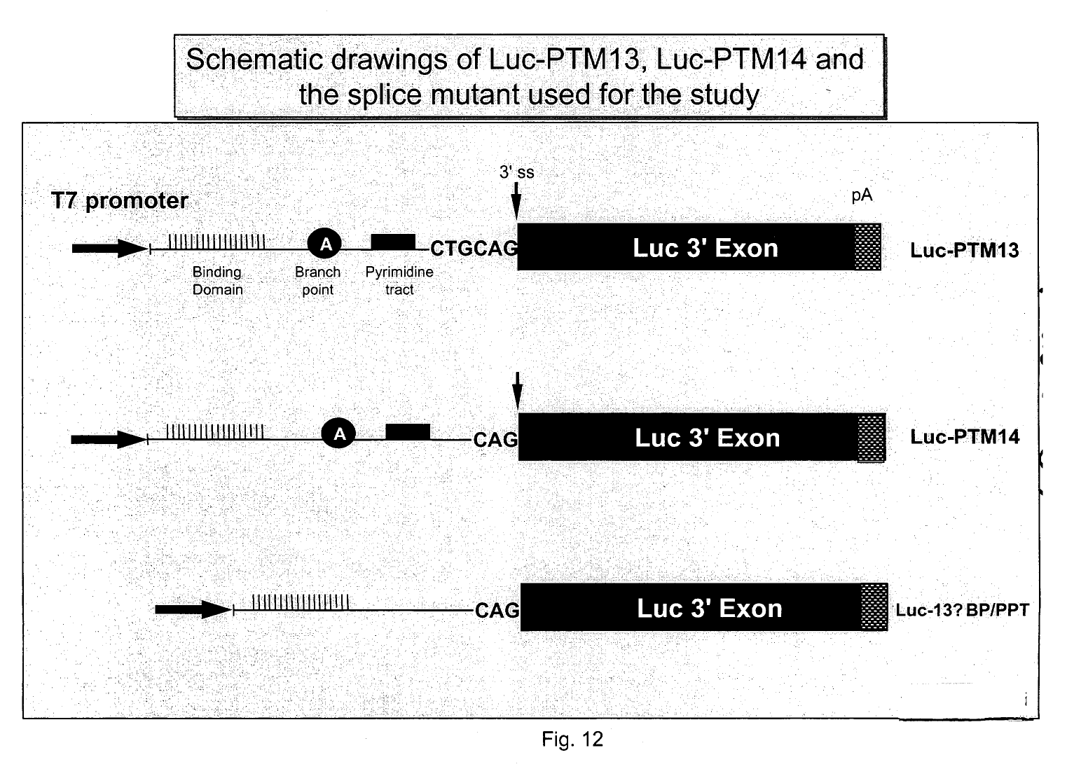

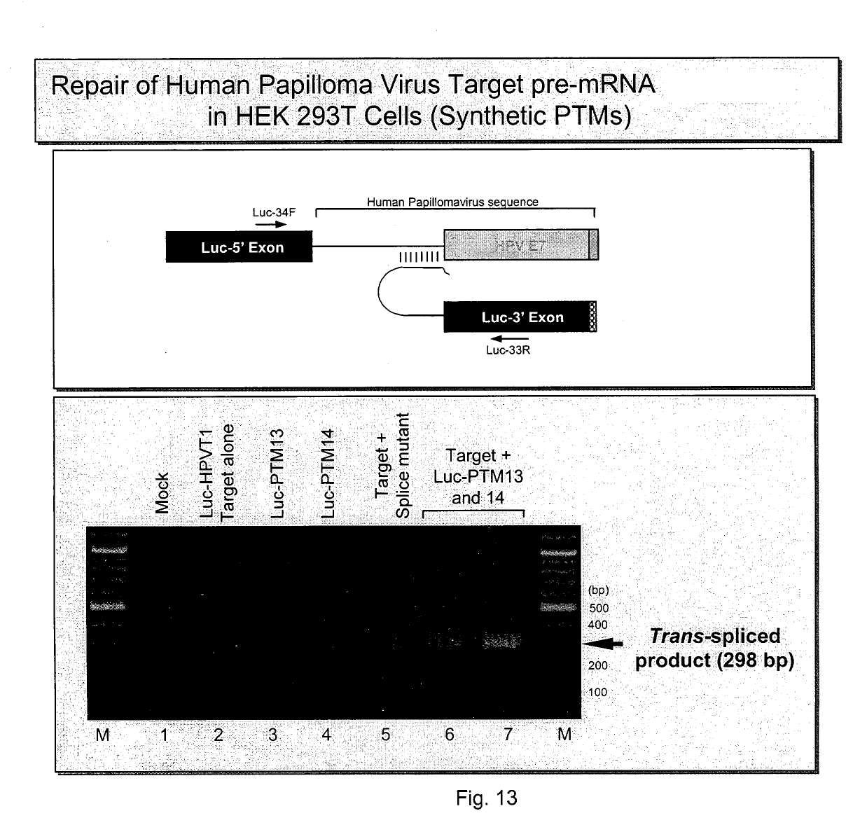

Using in vitro synthesized PTM RNA as genetic material, it was demonstrated that synthetic PTMs could be utilized for imaging of gene expression in human cells. As described above, to quantify the level of synthetic Renilla luciferase activity generated by trans-splicing, a synthetic Renilla luciferase model system was developed. (See FIG. 8). To demonstrate the use of synthetic PTMs, Luc-PTM13, Luc-PTM14, Luc-13 ABP/PPT (FIG. 12) and the HPV-Luciferase chimeric target (HPV-LucT1) RNAs (capped and poly A tailed) were synthesized using bacteriophage T7 RNA polymerase in vitro. Contaminating DNA was destroyed by treating with RNase free DNase 1, the RNA was purified and used for transfections. The transfections were performed as described above using DIMRE-C reagent. 48 hours post-transfection, total cellular RNA was isolated using MasterPure RNA isolation kit and analyzed by RT-PCR using target (Luc-34F) and PTM (Luc-33R) specific primers as described above. As shown in FIG. 13, no product was detected with RNA samples from mock, target or PTM alone control transfections (lanes 1-4). RNA from cells that were co-transfected with the HPV-luciferase target and a functional PTM produced a specific 298 bp product (FIG. 13, lanes 6 and 7). No such product was detected with RNA from cells that were co-transfected with target and splice mutant PTM (Luc13ΔPB/PPT), which does not contain a functional 3′ splice site (no branchpoint and polypyrimidine tract) (lane 5). These results not only demonstrate the importance of both the branchpoint and the pyrimidine tract for trans-splicing but also confirm that the production of the 298-bp product is due to trans-splicing. The accuracy of trans-splicing between HPV-LucT1 target pre-mRNA and Luc-PTM13 and Luc-PTM14 was confirmed by direct sequencing of the RT-PCR product.

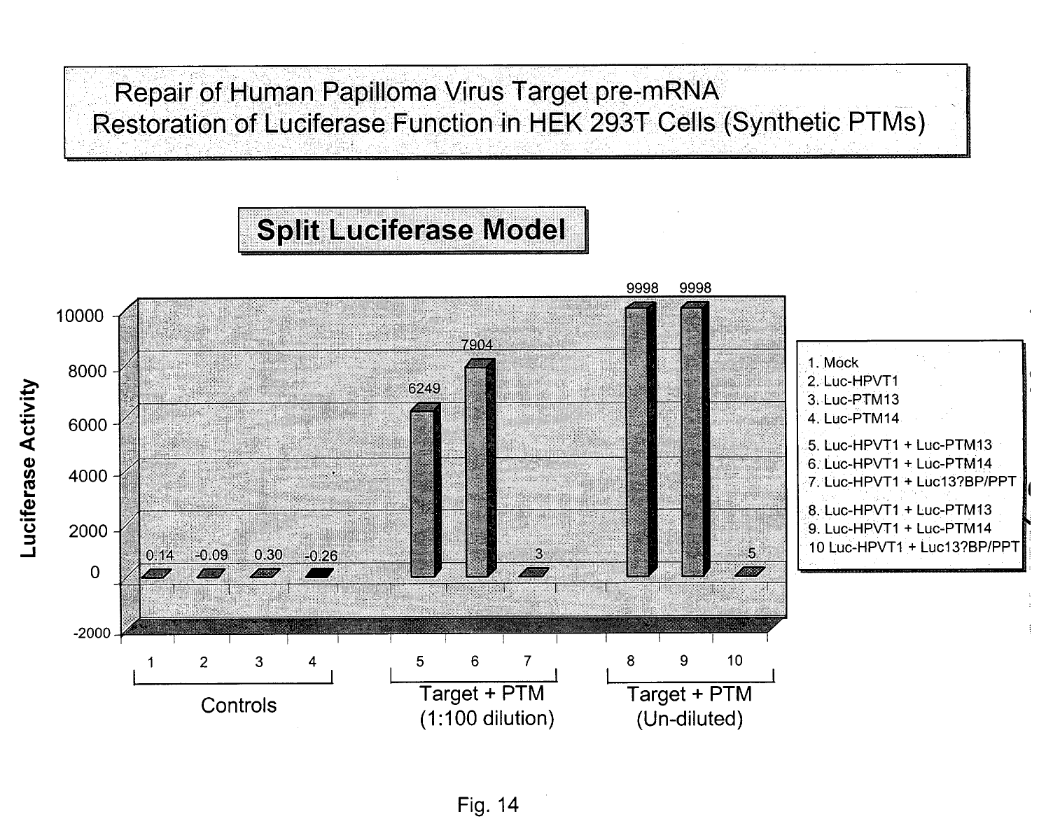

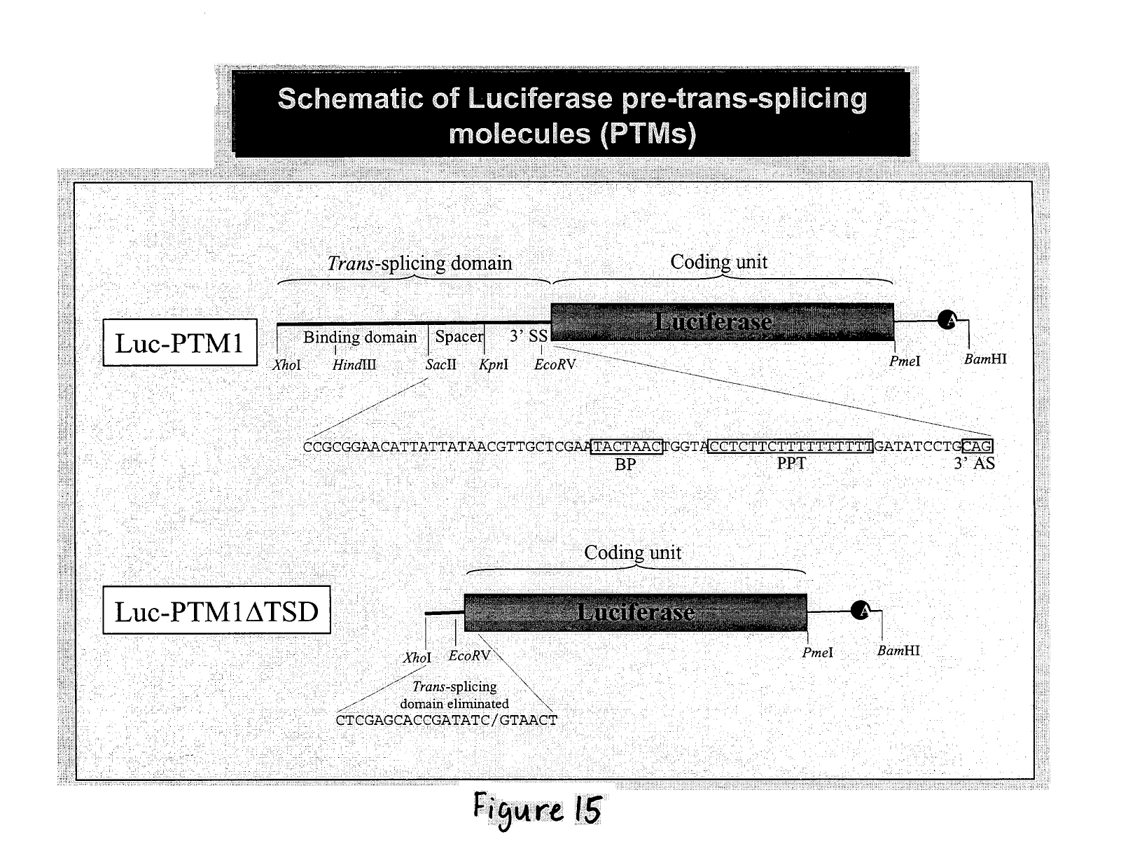

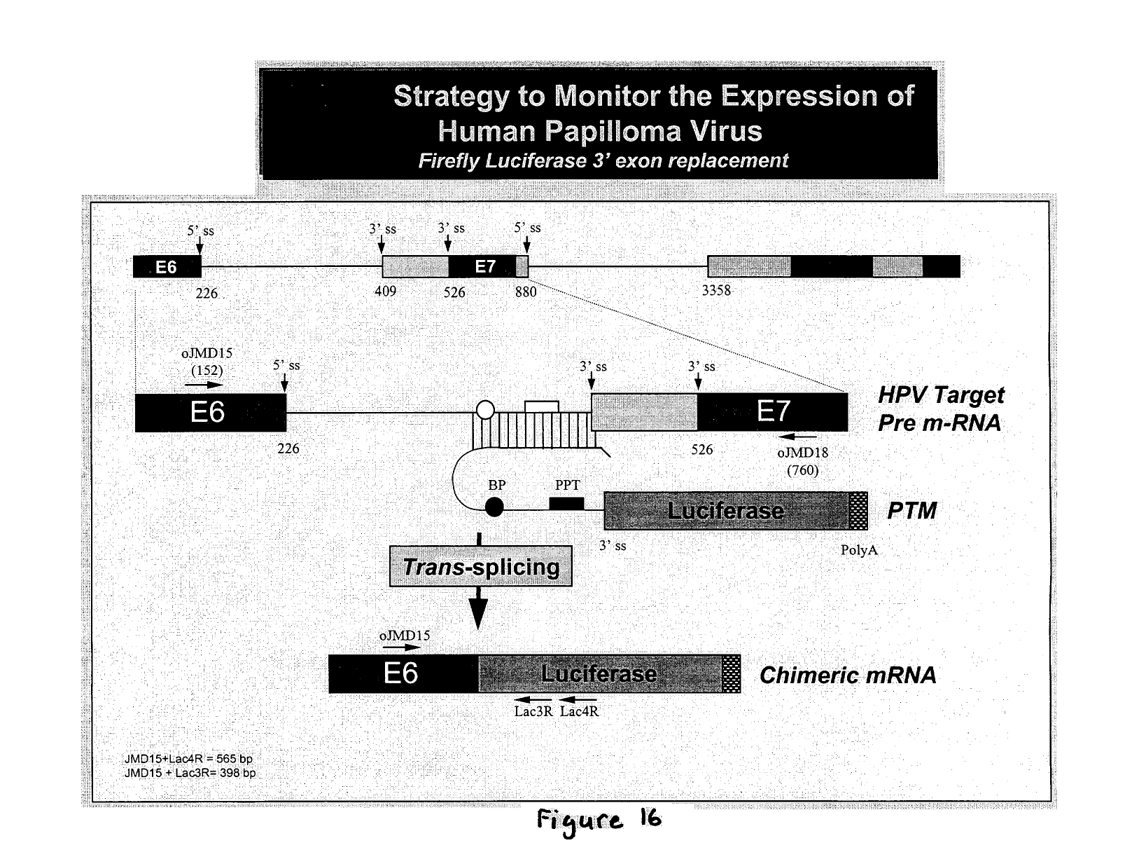

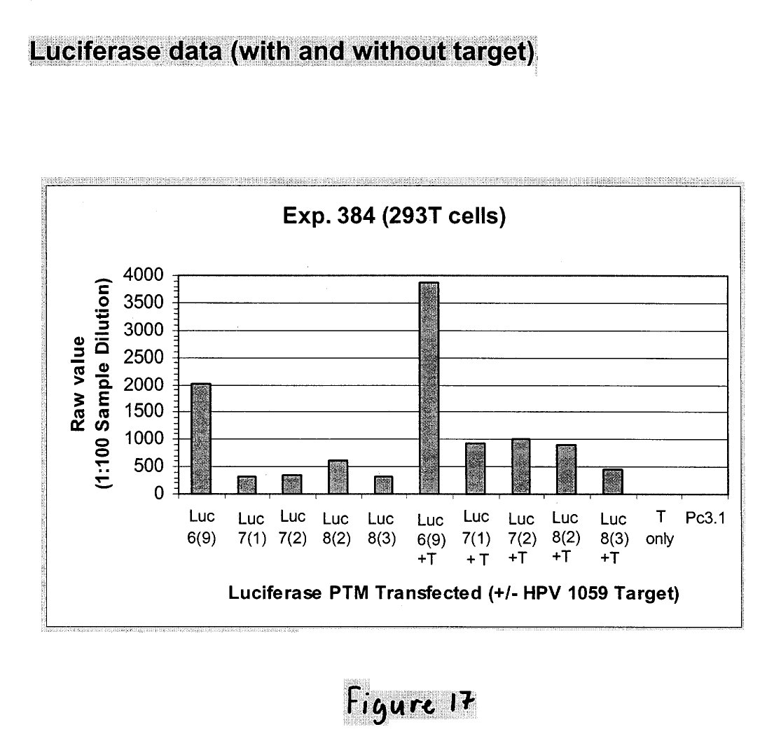

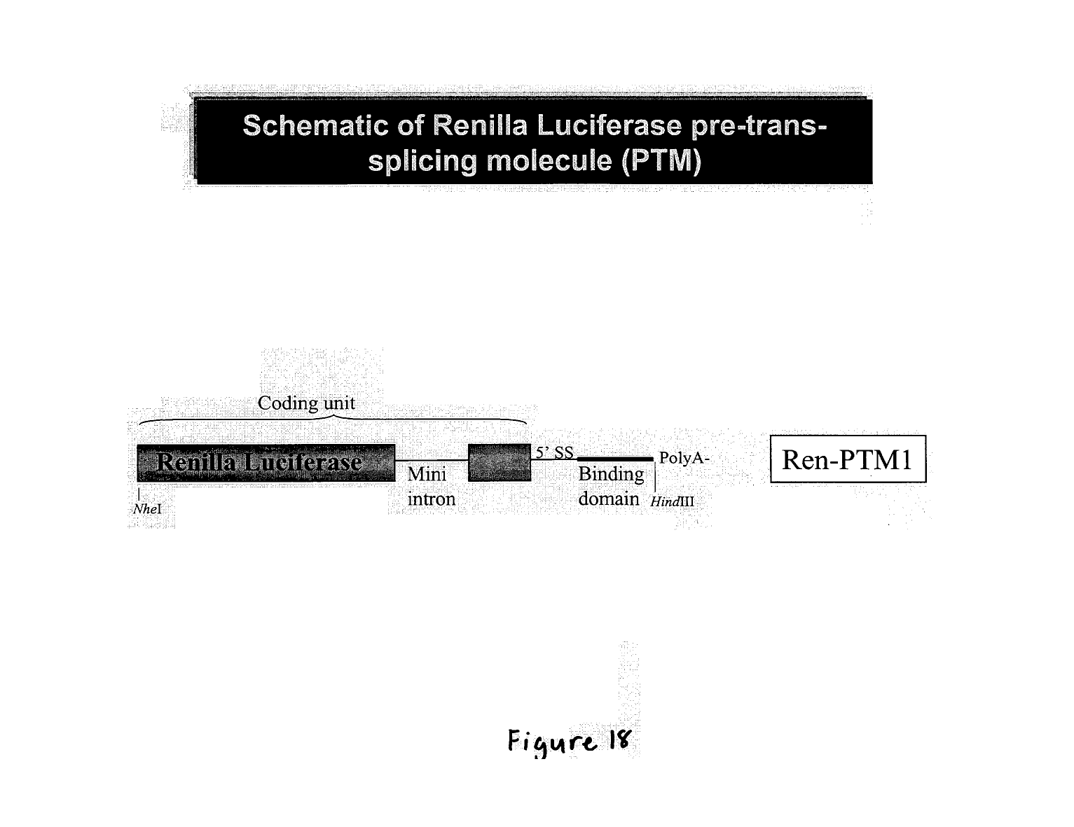

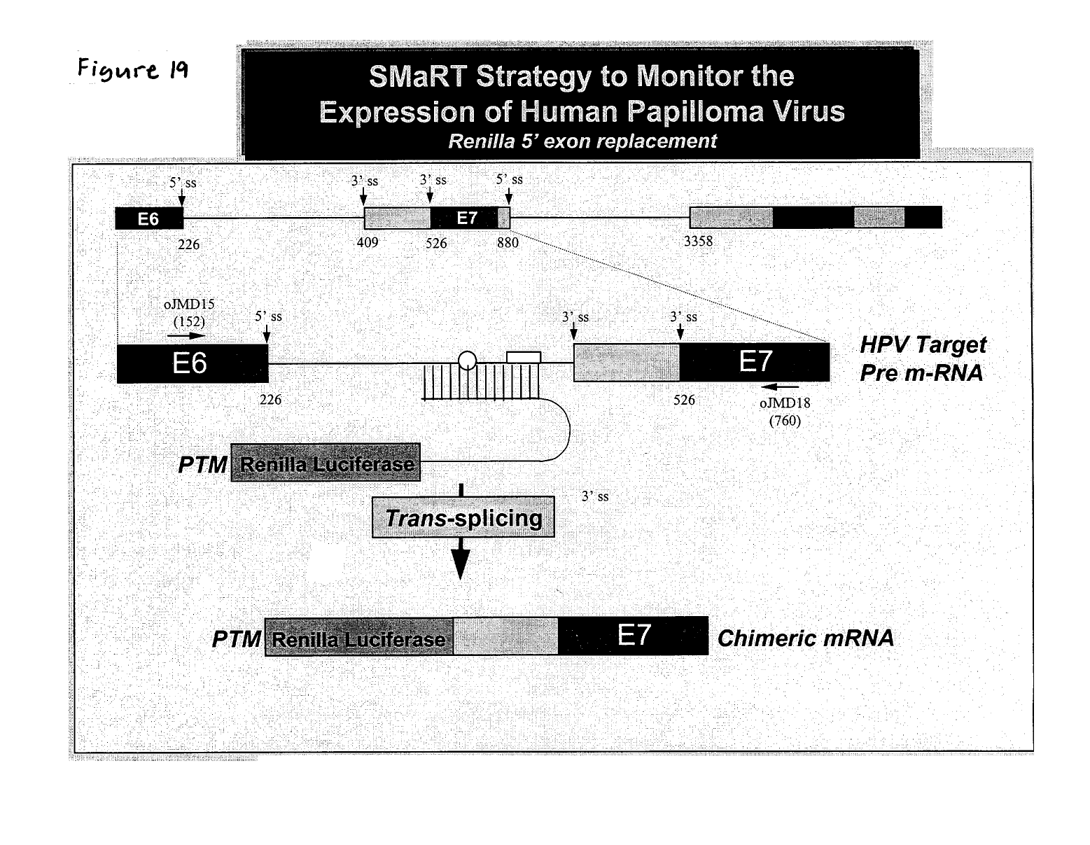

The efficiency of trans-splicing mediated mRNA repair and restoration of synthetic Renilla luciferase function was confirmed by assaying for enzymatic activity. As shown in FIG. 14, the synthetic Renilla luciferase activity in target or PTM alone control transfections is essentially at the background level that is observed in mock transfection. Co-transfection with a specific HPV-luciferase target (HPV-LucT1) along with Luc-PTM 13 or Luc-PTM 14 resulted in the repair of the target pre-mRNA and restored synthetic Renilla luciferase activity to a level that is 2000-fold over the background observed with a splice mutant PTM under similar experimental conditions. These results demonstrated the successful use of synthetic PTMs for imaging of gene expression.

### EXAMPLE 9

Imaging Through 3′ Exon Replacement

The PTM contains the complete coding of firefly luciferase minus the AUG start codon. The trans-splicing domain consists of a set of strong 3′ splice elements (including a yeast consensus branchpoint, a long pyrimidine tract and a 3′ acceptor site), a spacer sequence and a 125 nucleotide binding domain complementary to the,3′ end of the intron between exons E6 and E7 of human papilloma virus (HPV) (FIG. 15). The trans-splicing model for this PTM is shown in FIG. 16. To prevent PTM translation in the absence of trans-splicing a number of methionines in the 5′ end of the PTM coding were modified. This was carried out by site directed mutagenesis in which methionines were converted to codons that were considered conservative substitutions (based on amino acid alignments with other luciferase genes).

One potential problem is that in some instances the PTM itself may be translated. Since the 3′ exon replacement luciferase PTMs include the complete luciferase coding (minus the AUG initiator codon) and not a fragment of the full-length cDNA (as is the case with most previous PTMs) there could be a problem with un-spliced PTM being exported into cytoplasm and translation in the absence of trans-splicing. Thus, a Renilla luciferase based PTM that can perform 5′ exon replacement was generated. This form of PTM has the potential advantage of reduced PTM translation since the constructs can be engineered without a polyA signal. In the absence of this signal the RNA cannot be properly processed and translated.

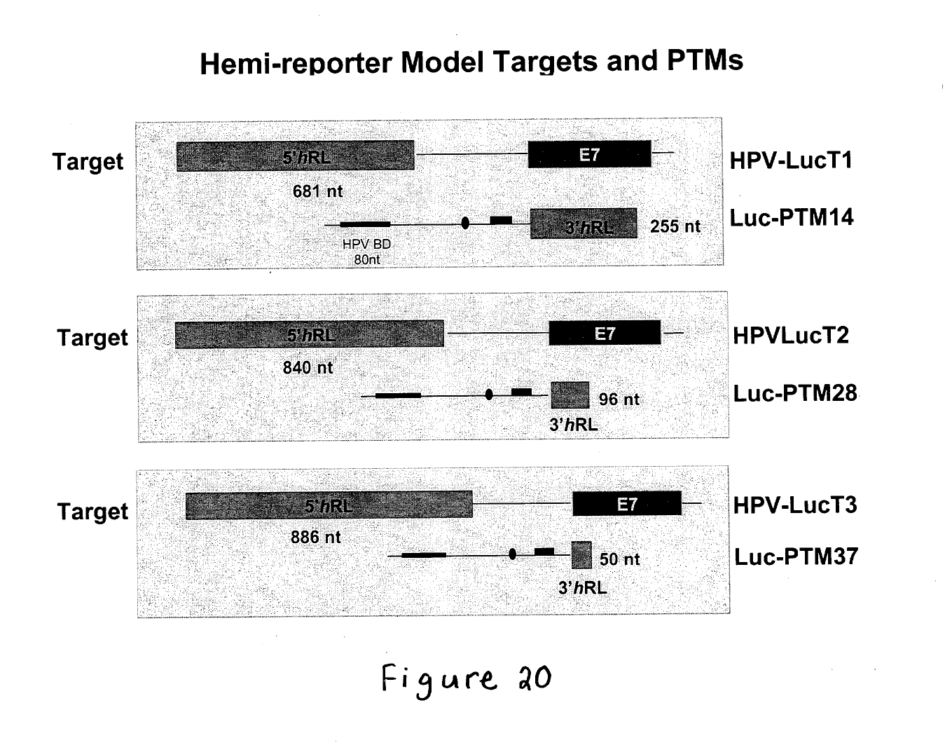

The structure of the Renilla luciferase 5′ exon replacement PTM is shown in FIG. 18. It consists of the full coding for Renilla luciferase split into two “exons”, separated by a mini-intron. The trans-splicing domain contains a consensus 5′ donor site, a short spacer sequence and a binding domain complementary to the 3′ end of the intron between exons E6 and E7 of the human papilloma virus (HPV). The trans-splicing model for this PTM is shown in FIG. 19.

Firefly luciferase PTMs were cotransfected with or without a HPV mini-gene target (see FIG. 16) in 293T cells. Cells were harvested after 48 hours and assayed for luciferase activity. These experiments showed that samples with target produced 2 fold higher activity indicating that trans-splicing was occurring with the mini-gene target and that there was reduced translation of the PTM (see FIG. 17).

### EXAMPLE

Hemi-Reporter Model Targets and PTMs

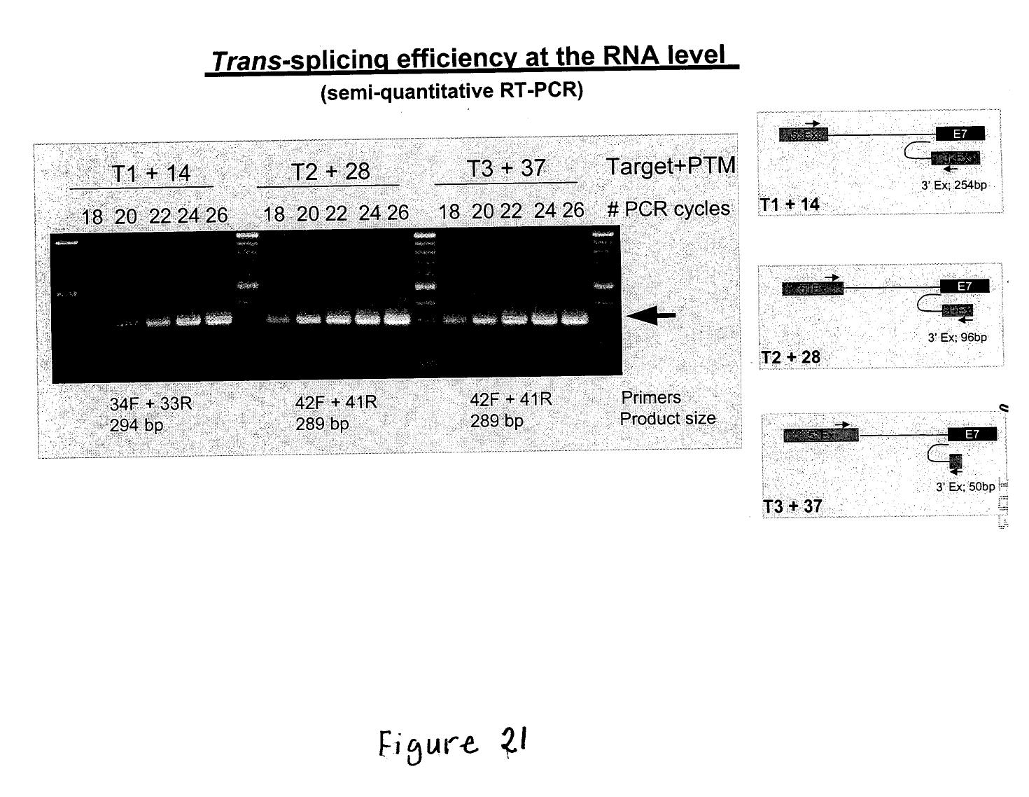

FIG. 20 depicts the hemi-reporter model targets and PTMs used for imaging of gene expression. The mini-gene pre-mRNA targets consists of the 5′ portion of humanized Renilla luciferase (hRluc) which acts as a “5′ exon”, coupled to the E6-E7 intron region and adjacent E7 coding sequence of human papilloma virus (HPV16).

As depicted in FIG. 20, PTMs consisting of the remainder of the reporter molecule were engineered to repair the mRNA and restore function. Several PTMs were constructed consisting of a “binding domain” complementary to the HPV target intron, a 3′ splice site (consisting of a BP, PPT and acceptor AG nucleotide), and the remainder hRL sequence as a 3′exon. The only difference between the PTMs is the “3′exon” size which ranged in size from 255 nt to 50 nt. Through its binding domain, the PTM is expected to base pair and co-localize with the target pre-mRNA. This facilitates trans-splicing between the splice sites of the target “5′ exon” and the “3′ exon” of the PTM, repairing the target mRNA and producing enzymatic activity.

To compare the trans-splicing efficiency of PTMI4, PTM28 and PTM37, human embryonic kidney (293T) cells were transfected with target and with the PTMs described above. 48 hours post-transfection, total cellular RNA was isolated and analyzed by RT-PCR using a target and a PTM specific primer. Based on a semi-quantitative estimation, Luc-PTM28 and Luc-PTM37 showed more efficient trans-splicing (˜2-4 fold) compared to Luc-PTM14 (FIG. 21). Here, a smaller PTMs trans-spliced more efficiently than the larger PTMs.

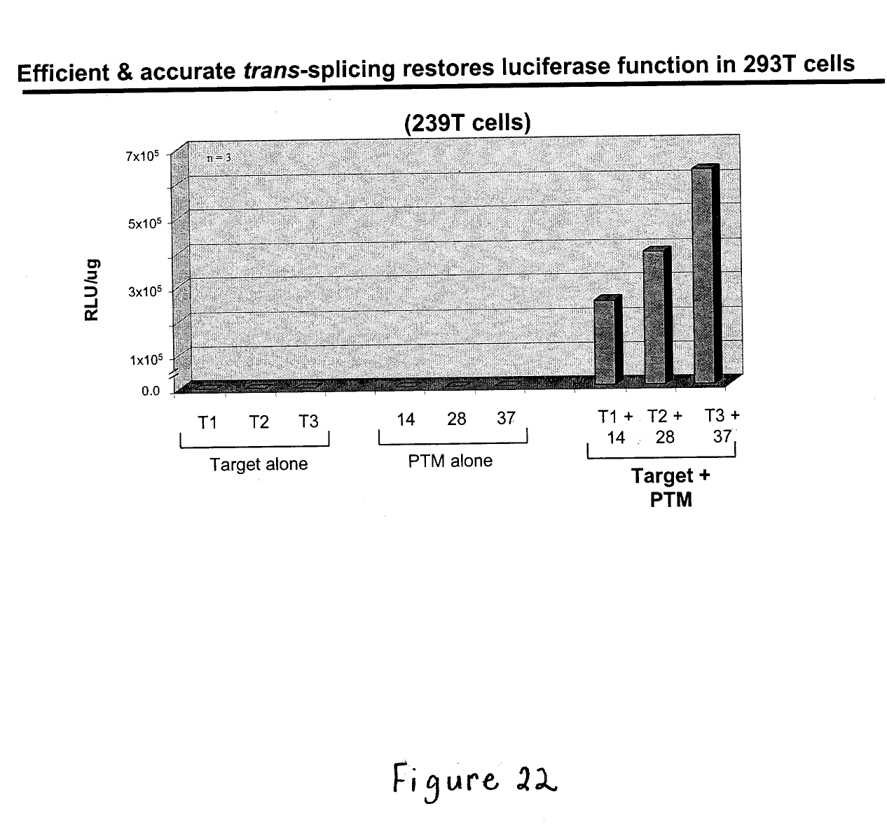

The efficiency of trans-splicing mediated mRNA repair and restoration of Luciferase function was confirmed by assaying for enzymatic activity. As demonstrated in FIG. 22, Luciferase activity in target or PTM alone control transfections is essentially at the background level that is observed in mock transfection. Co-transfection with a specific HPV-luciferase hemi-reporter target, HPV-LucT 1, HPV-LucT2 or HPV-LucT3 along with Luc-PTM14, Luc-PTM28 or Luc-PTM37, respectively, resulted in the efficient repair of pre-mRNA targets and restored luciferase activity on the order of 3-4 logs over background (FIG. 22). Luciferase activity produced by Luc-PTM37 is ˜3 fold higher compared to Luc-PTM14.

### EXAMPLE

Imaging of Gene Expression Using Full-Length Reporter PTMs

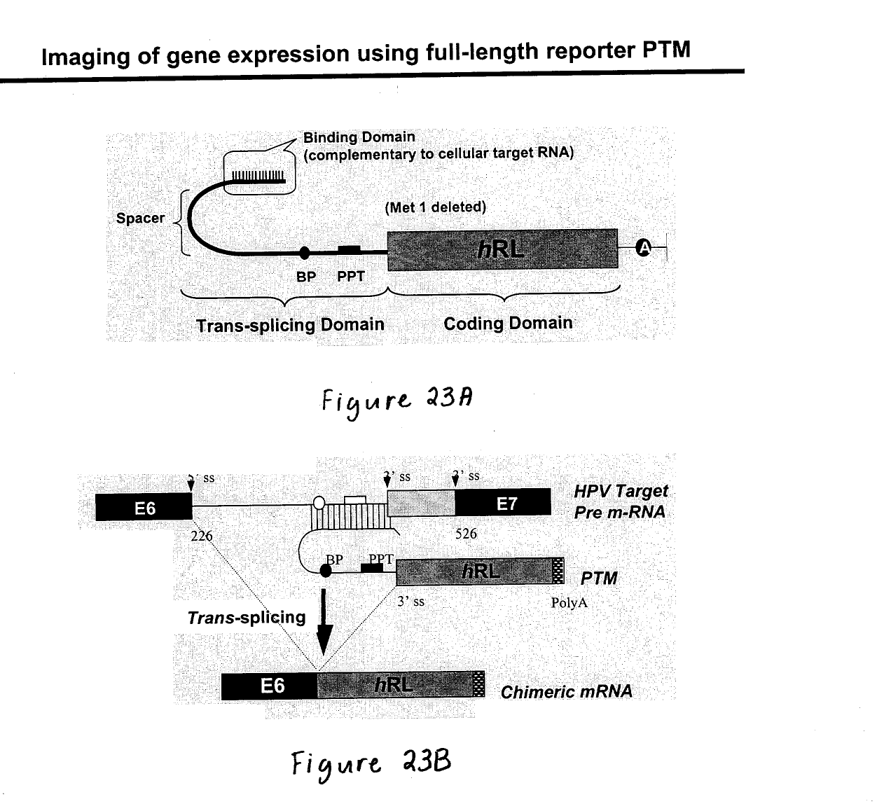

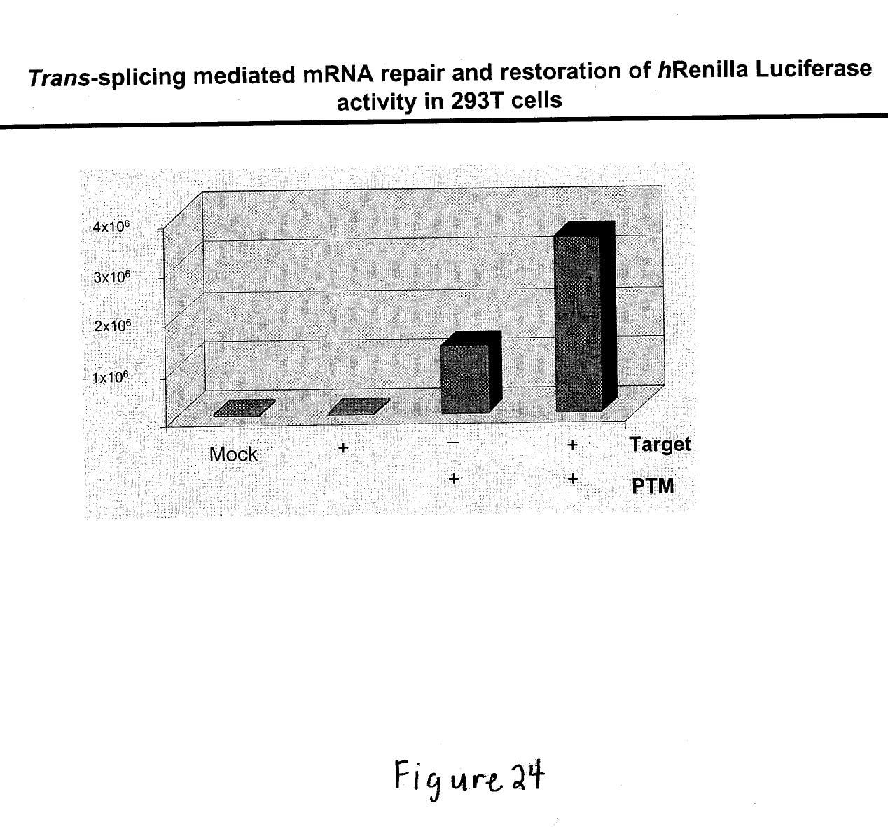

The full length imaging PTM (Luc-PTM27) contains the complete coding sequence for humanized Renilla Luciferase (hRL) minus the AUG start codon. The trans-splicing domain consists of a strong 3′ splice element (including a yeast consensus branch point (BP), a long pyrimidine tract (PPT) and a 3′ acceptor site), a spacer sequence and a 80 nucleotide binding domain (BD) complementary to the 3′ end of the intron between exons E6 and E7 of human papilloma virus (HPV-16) (FIG. 23A). Schematic illustration of trans-splicing mediated restoration of Luciferase function is shown in FIG. 23B.

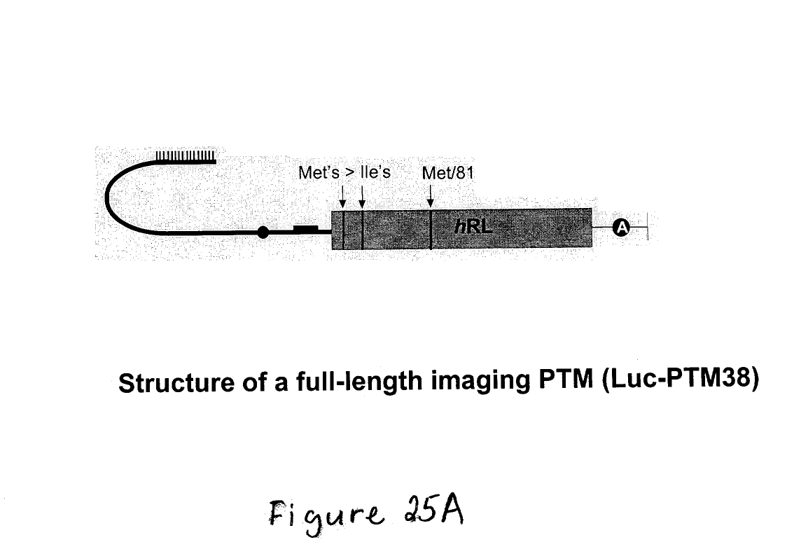

Full-length imaging PTM was co-transfected with or without a HPV mini-gene target into 293 cells. Cells were harvested after 48 hr of post-transfection and assayed for luciferase activity. The results depicted in FIG. 24 demonstrate that cells with target produced 3 fold higher luciferase activity indicating the proper trans-splicing between the HPV mini-gene target and the PTM. The results also indicate that this particular PTM (in the absence of target) does express the reporter which may be partly due to (i) direct translation of the PTM, (ii) PTM cis-splicing and translation or (iii) non-specific trans-splicing.

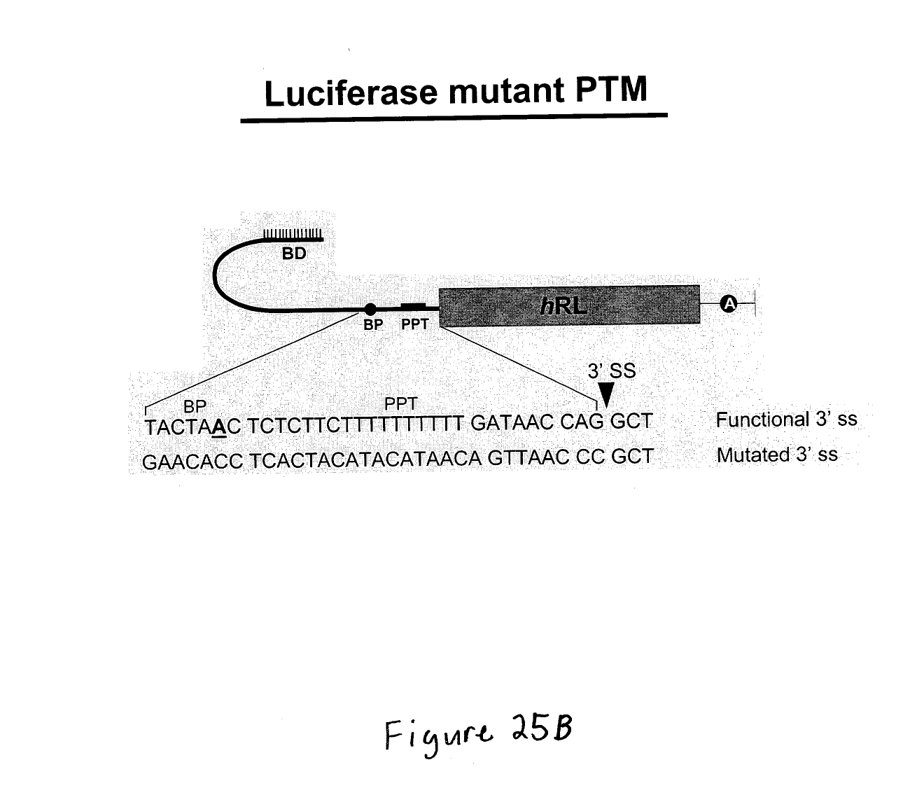

A Luciferase splice mutant PTM was constructed to determine whether the restoration of Luciferase function is due to RNA trans-splicing (FIG. 25B). The PTM is a derivative of Luc-PTM38 (FIG. 25A) in which the 3′ splice elements such as BP, PPT and the acceptor AG dinucleotide were modified by PCR mutagenesis and were confirmed by sequencing.

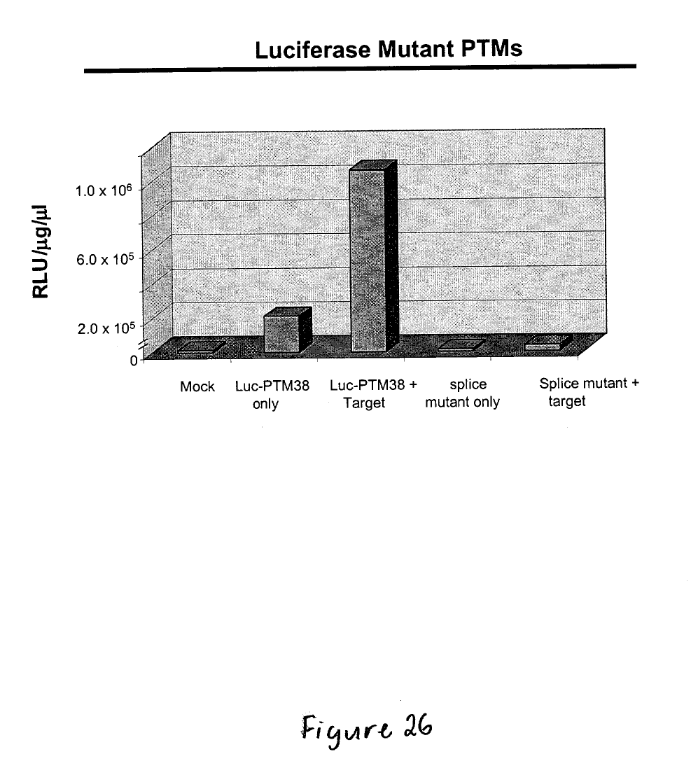

293T cells were co-transfected with or without HPV mini-gene target along with either a functional or splice mutant PTM. Cells were harvested after 48 hours and assayed for Luciferase function. As depicted in FIG. 26, the Luciferase activity in cells transfected with splice mutant PTM and with or without HPV mini-gene target are similar to the background observed with mock transfection. In contrast, cells that were co-transfected with Luc-PTM38 (functional PTM) and with target produced 4-5 fold more Luciferase activity compared to PTM38 alone.

### EXAMPLE

In Vivo Imaging of Gene Expression

Imaging approaches capable of detecting and quantitating levels of mRNA expression would be potentially useful for detecting the in vivo expression of genes including those associated with diseases such as infectious diseases and proliferative, neurological and metabolic disorders. The results described below demonstrate the successful in vivo detection of gene expression through spliceosome mediated RNA trans-splicing. The experimental results described below indicate the successful development of PTMs that can target and trans-splice reporter molecule sequences into an endogenous pre-mRNA of interest producing a chimeric mRNA encoding the reporter gene through spliceosome mediated RNA trans-splicing. This approach provides methods for indirect imaging of mRNA levels through imaging of a reporter protein. In contrast to imaging approaches that use antisense molecules to tatrget mRNA, spliceosome mediated RNA trans-splicing leads to direct signal amplification.

A pre-mRNA target was constructed that had the 5′ part of hRluc sequence, coupled to the coding sequence for human papilloma virus (HPV) E6 & E7 and the intronic sequences immediately upstream. Cis-splicing of HPV-LucT1 does not produce any hRluc activity. Several PTMs carrying the remaining hRluc sequence as a 3′ exon were genetically engineered. Through its targeting domain, the PTM base pairs with the HPV-LucT1 intron and trans-splices the 3′ luciferase exon, thereby repairing the pre-mRNA target and subsequently restoring enzymatic activity. The PTMs conatin a targeting domain that is complementary to the intron in HPV-LucT1, a branch point (BP) and pyrimidine tract (Py). For in vivo applications, PTMs were complexed with transferrinpolyethylineamine (Tf-PEI) (Hildebrandt, I. et al., 2002, Molecular Therapy 5:S421).

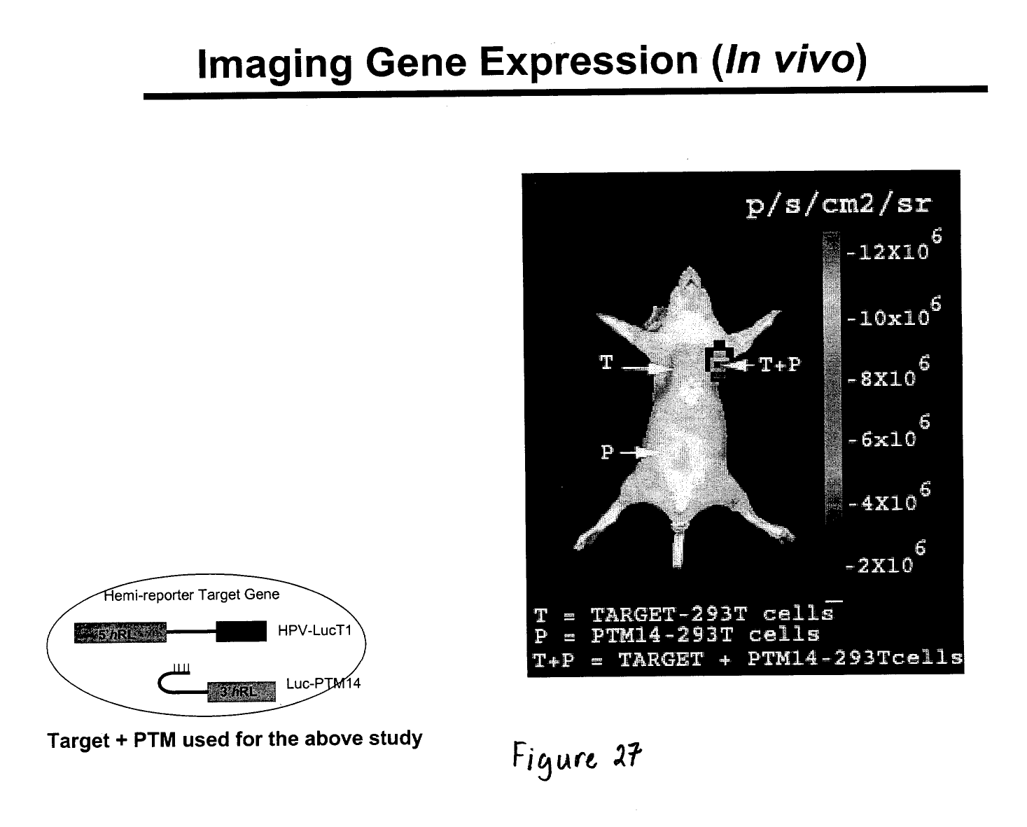

To test in vivo imaging of gene expression, 2.5×10⁶ 293T cells were transfected with PTM14, target or target+PTM14 (10 μg /plate) on Day 1. The ratio of PTM to target was 1:1. On Day 2, cells were washed with PBS and 1×10⁶ cells were injected subcutaneously into a mouse. On Day 3, cells were imaged immediately after injection of Coelenterazine substrate via tail vein using a cooled CCD camera (Bhaumik & Gambhir, 2002, Proc. Natl. Acad. Sci. USA 99:377-382). As depicted in FIG. 27, no signal was detected in cells transfected with target (T) or PTM (P) alone. In contrast, cells co-transfected with target and PTM produced high signal levels (T+P). The results clearly indicate successful RNA trans-splicing to image gene expression in vivo.

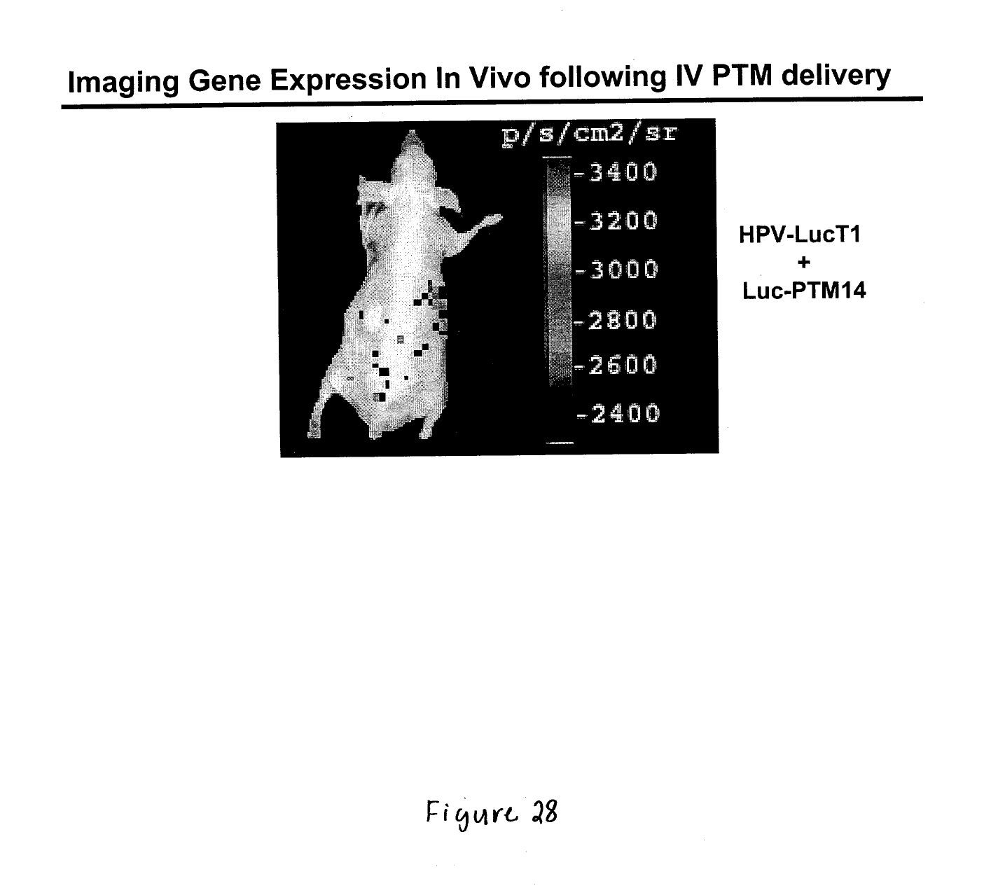

In a second experiment, 2.5×10⁶ N2a cells were transiently transfected with HPV-LucT1 target plasmid (10 μg) on Day 1. On Day 2, cells were washed with PBS and 5×10⁶ cells were implanted into 3-4 week old nude mice. Following implantation, 50 μg of Luc-PTM-14 conjugated with transferring-polyethylineamine (Tf-PEI) was then injected into the mouse via the tail vein. On Day 3, 80 μg of Coelenterazine substrate was injected via tail vein and the mice were imaged immediately for 5 min using a cooled CCD camera. As depicted in FIG. 28 tumors expressing HPV-LucT1 pre-mRNA target produced signals that were statisitically significant (P<0.05). In contrast, no signal was detected with N2a control tumor. The results depicted in FIG. 28 demonstrate imaging of gene expression in vivo following IV PTM delivery into target cells.

The present invention is not to be limited in scope by the specific embodiments described herein. Indeed, various modifications of the invention in addition to those described herein will become apparent to those skilled in the art from the foregoing description and accompanying Figures. Such modifications are intended to fall within the scope of the appended claims. Various references are cited herein, the disclosure of which are incorporated by reference in their entireties.

What is claimed:

1. A cell comprising a nucleic acid molecule wherein said nucleic acid molecule comprises:
a) one or more target binding domains that target binding of the nucleic acid molecule to a target pre-mRNA expressed within the cell;
b) a 3′ splice region comprising a branch point, a pyrimidine tract and a 3′ splice acceptor site;
c) a spacer region that separates the 3′ splice region from the target binding domain; and
d) a nucleotide sequence encoding a reporter molecule to be trans-spliced to the target pre-mRNA;
wherein said nucleic acid molecule is recognized by nuclear splicing components within the cell.

2. A cell comprising a nucleic acid molecule wherein said nucleic acid molecule comprises:
a) one or more target binding domains that target binding of the nucleic acid molecule to a target pre-mRNA expressed within the cell;
b) a 3′ splice acceptor site;
c) a spacer region that separates the 3′ splice region from the target binding domain; and
d) a nucleotide sequence encoding a reporter molecule to be trans-spliced to the target pre-mRNA;
wherein said nucleic acid molecule is recognized by nuclear splicing components within the cell.

3. A cell comprising a nucleic acid molecule wherein said nucleic acid molecule comprises:
a) one or more target binding domains that target binding of the nucleic acid molecule to a target pre-mRNA expressed within the cell;
b) a 5′ splice site;
c) a spacer region that separates the 5′ splice site from the target binding domain; and
d) a nucleotide sequence to be trans-spliced to the target pre-mRNA;
wherein said nucleic acid molecule is recognized by nuclear splicing components within the cell.

4. The cell of claim 1 wherein the nucleic acid molecule further comprises a 5′ donor site.

5. The cell of claim 1, 2, 3, or 4 wherein the nucleic acid molecule further comprises a safety nucleotide sequence comprising one or more complementary sequences that bind to one or more sides of the 3′ or 5′ splice regions.

6. The cell of claim 1, 2, 3, 4 or 5 wherein the reporter molecule provides a fluorescent signal.

7. The cell of claim 1, 2, 3, 4 or 5 wherein the reporter molecule provides a bioluminescent signal.

8. The cell of claim 1, 2, 3, 4 or 5 wherein the reporter molecule provides a radioactive signal.

9. The cell of claim 1, 2, 3, 4 or 5 wherein the reporter molecule is selected from the group consisting of (i) an enzyme; (ii) a receptor; (iii) a protein; (iv) a peptide tag; (iv) an ion channel; or (v) antibody.

10. The cell of claim 1, 2, 3, 4 or 5 wherein the reporter molecule is has an affinity for a labeled probe or tracer.

11. The cell of claim 1, 2, 3, 4 or 5 wherein the target pre-mRNA is derived from an infectious agent.

12. The cell of claim 1, 2, 3, 4 or 5 wherein the target pre-mRNA is derived from a virus.

13. The cell of claim 1, 2, 3, 4 or 5 wherein expression of the target pre-mRNA is associated with a proliferative disorder.

14. A method of producing a chimeric RNA molecule in a cell wherein said chimeric molecule encodes a reporter molecule comprising:contacting a target pre-mRNA expressed in the cell with a nucleic acid molecule recognized by nuclear splicing components wherein said nucleic acid molecule comprises:
a) one or more target binding domains that target binding of the nucleic acid molecule to a target pre-mRNA expressed within the cell;
b) a 3′ splice region comprising a branch point, a pyrimidine tract and a 3′ splice acceptor site;
c) a spacer region that separates the 3′ splice region from the target binding domain; and
d) a nucleotide sequence encoding a reporter molecule to be trans-spliced to the target pre-mRNA; under conditions in which a portion of the nucleic acid molecule is trans-spliced to a portion of the target pre-mRNA to form a chimeric RNA within the cell.

15. A method of producing a chimeric RNA molecule in a cell wherein said chimeric molecule encodes a reporter molecule comprising:contacting a target pre-mRNA expressed in the cell with a nucleic acid molecule recognized by nuclear splicing components wherein said nucleic acid molecule comprises:
a) one or more target binding domains that target binding of the nucleic acid molecule to a target pre-mRNA expressed within the cell;
b) a 3′ splice acceptor site;
c) a spacer region that separates the 3′ splice region from the target binding domain; and
d) a nucleotide sequence encoding a reporter molecule to be trans-spliced to the target pre-mRNA; under conditions in which a portion of the nucleic acid molecule is trans-spliced to a portion of the target pre-mRNA to form a chimeric RNA within the cell.

16. A method of producing a chimeric RNA molecule in a cell wherein said chimeric molecule encodes a reporter molecule comprising:
contacting a target pre-mRNA expressed within the cell with a nucleic acid molecule recognized by nuclear splicing components wherein said nucleic acid molecule comprises:
a) one or more target binding domains that target binding of the nucleic acid molecule to a target pre-mRNA expressed within the cell;
b) a 5′ splice site;
c) a spacer region that separates the 5′ splice site from the target binding domain; and
d) a nucleotide sequence encoding a reporter molecule to be trans-spliced to the target pre-mRNA; wherein a chimeric RNA molecule is produced within the cell.

17. A method of claim 14 wherein the nucleic acid molecule further comprises a 5′ donor site.

18. The cell of claim 14, 15, 16, or 17 wherein the nucleic acid molecule further comprises a safety nucleotide sequence comprising one or more complementary sequences that bind to one or more sides of the 3′ or 5′ splice regions.

19. The cell of claim 14, 15, 16, or 17 wherein the reporter molecule provides a fluorescent signal.

20. The cell of claim 14, 15, 16, or 17 wherein the reporter molecule provides a bioluminescent signal.

21. The cell of claim 14, 15, 16, or 17 wherein the reporter molecule provides a radioactive signal.

22. The cell of claim 14, 15, 16, or 17 wherein the reporter molecule is selected from the group consisting of (i) an enzyme; (ii) a receptor; (iii) a protein; (iv) a peptide tag; (iv) an ion channel; or (v) antibody.

23. The cell of claim 14, 15, 16, or 17 wherein the reporter molecule is has an affinity for a labeled probe or tracer.

24. The cell of claim 14, 15, 16, or 17 wherein the target pre-mRNA is derived from an infectious agent.

25. The cell of claim 14, 15, 16, or 17 wherein the target pre-mRNA is derived from a virus.

26. The cell of claim 14, 15, 16, or 17 wherein expression of the target pre-mRNA is associated with a proliferative disorder.

27. A nucleic acid molecule comprising:
a) one or more target binding domains that target binding of the nucleic acid molecule to a target pre-mRNA expressed within a cell;
b) a 3′ splice region comprising a branch point, a pyrimidine tract and a 3′ splice acceptor site;
c) a spacer region that separates the 3′ splice region from the target binding domain; and
d) a nucleotide sequence encoding a reporter molecule to be trans-spliced to the target pre-mRNA;
wherein said nucleic acid molecule is recognized by nuclear splicing components within the cell.

28. A nucleic acid molecule comprising:
a) one or more target binding domains that target binding of the nucleic acid molecule to a target pre-mRNA expressed within a cell;
b) a 3′ splice acceptor site;
c) a spacer region that separates the 3′ splice region from the target binding domain; and
d) a nucleotide sequence encoding a reporter molecule to be trans-spliced to the target pre-mRNA;
wherein said nucleic acid molecule is recognized by nuclear splicing components within the cell.

29. A nucleic acid molecule comprising:
a) one or more target binding domains that target binding of the nucleic acid molecule to a target pre-mRNA expressed within a cell;
b) a 5′ splice site;
c) a spacer region that separates the 5′ splice site from the target binding domain; and
d) a nucleotide sequence encoding a reporter molecule to be trans-spliced to the target pre-mRNA;
wherein said nucleic acid molecule is recognized by nuclear splicing components within the cell.

30. The nucleic acid molecule of claim 27 wherein the nucleic acid molecule further comprises a 5′ donor site.

31. The nucleic acid molecule of claim 27 further comprising a safety nucleotide sequence comprising one or more complementary sequences that bind to one or more sides of the 3′ or 5′ splice regions.

32. The nucleic acid molecule of claim 28, 29, 30 or 31 wherein the reporter molecule provides a fluorescent signal.

33. The nucleic acid molecule of claim 28, 29, 30 or 31 wherein the reporter molecule provides a bioluminescent signal.

34. The nucleic acid molecule of claim 28, 29, 30 or 31 wherein the reporter molecule provides a radioactive signal.

35. The nucleic acid molecule of claim 28, 29, 30 or 31 wherein the reporter molecule is selected from the group consisting of (i) an enzyme; (ii) a receptor; (iii) a protein; (iv) a peptide tag; (iv) an ion channel; or (v) antibody.

36. The nucleic acid molecule of claim 28, 29, 30 or 31 wherein the reporter molecule is has an affinity for a labeled probe or tracer.

37. The nucleic acid molecule of claim 28, 29, 30 or 31 wherein the target pre-mRNA is derived from an infectious agent.

38. The nucleic acid molecule of claim 28, 29, 30 or 31 wherein the target pre-mRNA is derived from a virus.

39. The nucleic acid molecule of claim 28, 29, 30 or 31 wherein expression of the target pre-mRNA is associated with a proliferative disorder.

40. A method for detecting the expression of a target pre-mRNA in a host cell comprising (i) contacting said host cell with a nucleic acid molecule wherein said nucleic acid molecule comprises:
a) one or more target binding domains that target binding of the nucleic acid molecule to a target pre-mRNA expressed within the cell;
b) a 3′ splice region comprising a branch point, a pyrimidine tract and a 3′ splice acceptor site;
c) a spacer region that separates the 3′ splice region from the target binding domain; and
d) a nucleotide sequence encoding a reporter molecule to be trans-spliced to the target pre-mRNA; wherein said nucleic acid molecule is recognized by nuclear splicing components within the cell; and
(ii) detecting expression of the reporter molecule.

41. A method for detecting the expression of a target pre-mRNA in a host cell comprising (i) contacting said host cell with a nucleic acid molecule wherein said nucleic acid molecule comprises:
a) one or more target binding domains that target binding of the nucleic acid molecule to a target pre-mRNA expressed within the cell;
b) a 3′ splice acceptor site;
c) a spacer region that separates the 3′ splice region from the target binding domain; and
d) a nucleotide sequence encoding a reporter molecule to be trans-spliced to the target pre-mRNA; wherein said nucleic acid molecule is recognized by nuclear splicing components within the cell; and
(ii) detecting expression of the reporter molecule.

42. A method for detecting the expression of a target pre-mRNA in a host cell comprising (i) contacting said host cell with a nucleic acid molecule wherein said nucleic acid molecule comprises:
a) one or more target binding domains that target binding of the nucleic acid molecule to a target pre-mRNA expressed within the cell;
b) a 5′ splice site;
c) a spacer region that separates the 5′ splice site from the target binding domain; and
d) a nucleotide sequence to be trans-spliced to the target pre-mRNA;wherein said nucleic acid molecule is recognized by nuclear splicing components within the cell; and
(ii) detecting expression of the reporter molecule.

45. The method of claim 40 wherein the nucleic acid molecule further comprises a 5′ donor site.

46. The method of claim 40 wherein the nucleic acid molecule further comprises a safety nucleotide sequence comprising one or more complementary sequences that bind to one or more sides of the 3′ or 5′ splice region.

47. The method of claim 40, 41, 42, 43 or 45 wherein the reporter molecule provides a fluorescent signal.

48. The method of claim 40, 41, 42, 43 or 45 wherein the reporter molecule provides a bioluminescent signal.

49. The method of claim 40, 41, 42, 43 or 45 wherein the reporter molecule provides a radioactive signal.

50. The method of claim 40, 41, 42, 43 or 45 wherein the reporter molecule is selected from the group consisting of (i) an enzyme; (ii) a receptor; (iii) a protein; (iv) a peptide tag; (iv) an ion channel; or (v) antibody.

51. The method of claim 40, 41, 42, 43 or 45 wherein the reporter molecule is has an affinity for a labeled probe or tracer.

52. The method of claim 40, 41, 42, 43 or 45 wherein the target pre-mRNA is derived from an infectious agent.

53. The method of claim 40, 41, 42, 43 or 45 wherein the target pre-mRNA is derived from a virus.

54. The method of claim 40, 41, 42, 43 or 45 wherein expression of the target pre-mRNA is associated with a proliferative disorder.


SEQUENCE LISTING (13 sequences)

SEQ ID NO: 1 - RNA, 8 nt, ARTIFICIAL SEQUENCE
  PTM 5′ SPLICE CONSENSUS SEQUENCE
agguragu

SEQ ID NO: 2 - RNA, 7 nt, ARTIFICIAL SEQUENCE
  PTM 3′CONSENSUS SEQUENCE
ynyurac

SEQ ID NO: 3 - RNA, 7 nt, ARTIFICIAL SEQUENCE
  PTM CONSENSUS BRANCHPOINT SEQUENCE
uacuaac

SEQ ID NO: 4 - DNA, 8 bp, ARTIFICIAL SEQUENCE
  GT RICH CONSENSUS SEQUENCE
ygtgttyy

SEQ ID NO: 5 - DNA, 29 bp, ARTIFICIAL SEQUENCE
  OLIGONUCLEOTIDE PRIMER
aagcttttactgctcgttcttcagcacgc

SEQ ID NO: 6 - DNA, 21 bp, ARTIFICIAL SEQUENCE
  OLIGONUCLEOTIDE PRIMER
cagggtcgggactcgatgaac

SEQ ID NO: 7 - DNA, 21 bp, ARTIFICIAL SEQUENCE
  OLIGONUCLEOTIDE PRIMER
ggatatcgccctgatcaagag

SEQ ID NO: 8 - DNA, 6 bp, ARTIFICIAL SEQUENCE
  PTM SEQUENCE
gctagc

SEQ ID NO: 9 - DNA, 6 bp, ARTIFICIAL SEQUENCE
  PTM SEQUENCE
ccgcgg

SEQ ID NO: 10 - DNA, 48 bp, ARTIFICIAL SEQUENCE
  PTM BRANCHPOINT AND POLYPYRIMIDINE TRACT SEQUENCES
tactaactggtaccgtcttcttttttttttgatatcctgcagggcggc

SEQ ID NO: 11 - DNA, 66 bp, ARTIFICIAL SEQUENCE
  TRANSPLICED PRODUCT
ctcctggcctcgcgagatccctctcgttaagggaggcaagcccgacgtcgtccagattgtccgcaa

SEQ ID NO: 12 - DNA, 71 bp, ARTIFICIAL SEQUENCE
  LUCPTM1 SPACER, BRANCHPOINT, AND POLYPYRIMIDINE TRACT SEQUENCES
ccgcggaacattattataacgttgctcgaatactaactggtacctcttcttttttttttgatatcctgcag

SEQ ID NO: 13 - DNA, 22 bp, ARTIFICIAL SEQUENCE
  LUCPTM1 DELTA TSD SEQUENCES
ctcgagcaccgatatcgtaact